(19) United States
(12) Patent Application Publication
(10) Pub. No.: US 2023/0212673 A1
(43) Pub. Date: Jul. 6, 2023

(54) LYMPHOCYTE CLONALITY DETERMINATION

(71) Applicant: SiMsen Diagnostics AB, Göteborg (SE)

(72) Inventor: Anders STÅHLBERG, Kållered (SE)

(21) Appl. No.: 18/001,080

(22) PCT Filed: Jun. 2, 2021
(86) PCT No.: PCT/SE2021/050517

§ 371 Date: Dec. 8, 2022

(30) Foreign Application Priority Data
Jun. 9, 2020 (SE) 2050673-9

(51) Int. Cl. C12Q 1/6881

(52) U.S. Cl. CPC C12Q 1/6881; C12Q 2600/16; C12Q 1/6858

(57) ABSTRACT

The present invention determines lymphocyte clonality by contacting a sample comprising nucleic acid molecules (1) of lymphocytes with forward and reverse primers (10, 20) and amplifying the nucleic acid molecules (1) by performing PCR pre-amplification to form barcoded PCR products (50). The barcoded PCR products (50) are amplified using adapter-specific forward and reverse primers (30, 40) in a PCR application into amplified barcoded PCR products (60), which are sequenced. The sequence reads are demultiplexed, mapped to respective TCR or BCR clonotypes and used to determine lymphocyte clonality for the sample. The forward and/or reverse primers (10, 20) are barcoded by comprising UMIs (14, 24) protected inside hairpin loops.

### TECHNICAL FIELD

The present embodiments generally relate to lymphocyte clonalities and, in particular to determining lymphocyte clonality.

### BACKGROUND

T and B lymphocytes undergo clonal expansion upon activation. The monitoring of clonality has immense importance in the diagnosis and follow-up of hematological diseases and is a fundamental tool for the understanding of antigen specificity in immunology applications, including infections, cancer, and in autoimmunity. Today, a comprehensive assessment of clonality is feasible using next-generation sequencing (NGS) approaches. Clinically, there is a need for improved methodological specificity and sensitivity to determine the immune repertoire, for example, to detect acute lymphoblastic leukemia and to monitor minimal residual disease. Moreover, in studies of immune disorders, such as autoimmunity and tumor immunity, improved immune repertoire analysis will facilitate the identification of involved immune cells, be used to evaluate the efficiency of immunomodulating drugs, and explore their mode of action.

T cells comprise T cell receptors (TCRs), which can either be constructed from the two subunits a and β or γ and δ. These subunits are composed from distinct genetic regions known as variable (V) and joining (J) regions, some subunits also include one or two diversity (D) regions. During immune cell maturation regions are randomly recombined on DNA level in the order variable, diversity, joining and in between each segment random addition and/or deletion of nucleotides take place, which construct a highly diverse and unique sequence per cell. The recombined sequence encodes a TCR with a unique antigen specificity and is inherited when the cell is clonally expanded. This is of interest when studying immune defense dynamics and other biological applications.

Correspondingly, B cells comprise B-cell receptors (BCRs) composed of immunoglobulin molecules forming a type 1 transmembrane receptor protein. The BCR consists of an antigen-binding subunit known as the membrane immunoglobulin (mIg), which is composed of two immunoglobulin light chains (IgLs) and two immunoglobulin heavy chains (IgHs) as well as two heterodimer subunits of Ig-α and Ig-β. Within the BCR, the part that recognizes antigens is composed of distinct V, D and J genetic regions similar to TCRs of T cells.

There are two principally different approaches to study the immune repertoire using NGS, targeted amplification of the third complementary determining region (CDR3) in DNA, or of the transcribed mRNA. A challenge when analyzing cell clonality with these standard NGS-based approaches is that the exact number and size of clones cannot be determined due to uneven amplification. Furthermore, clonotypes with similar sequences cannot be reliably distinguished from sequence errors introduced during PCR and sequencing. Several methods have been developed to overcome these limitations, including the use of synthetic spiked molecules to correct for quantification biases and bioinformatical approaches to reduce PCR-induced errors.

U.S. Pat. No. 9,394,567 discloses methods for detecting and quantifying nucleic acid contamination in a tissue sample of an individual containing T cells and/or B cells, which is used for generating a sequence-based clonotype profile. The method is implemented by measuring the presence and/or level of an endogenous or exogenous nucleic acid tag by which nucleic acid from an intended individual can be distinguished from that of unintended individuals. Endogenous tags include genetic identity markers, such as short tandem repeats, rare clonotypes or the like, and exogenous tags include sequence tags employed to determine clonotype sequences from sequence reads.

U.S. Pat. No. 9,506,119 discloses the use of sequence tags to improve sequence determination of amplicons of related sequences. Sequence reads having the same sequence tags are aligned after which final base calls are determined from an average base call from sequence read base calls at each position. Similarly, sequence reads comprising series of incorporation signals are aligned by common sequence tags and base calls in homopolymer regions are made as a function incorporation signal values at each “flow” position.

### SUMMARY

It is a general objective to provide a method for lymphocyte clonality determination.

It is a particular objective to provide such a method capable of identifying and quantifying TCR and/BCR clonotypes in a sample.

These and other objectives are met by embodiments as disclosed herein.

The present invention is defined in the independent claim. Further embodiments are defined in the dependent claims.

An aspect of the invention relates to a method of determining lymphocyte clonality. The method comprises contacting a sample comprising nucleic acid molecules of lymphocytes with M forward primers and N reverse primers. M is an integer equal to or larger than one and N is an integer equal to or larger than one. The M forward primers comprise, from a 5′ end to a 3′ end, an adapter sequence and a target-specific sequence. The N reverse primers comprise, from a 5′ end to a 3′ end, an adapter sequence and a target-specific sequence. The M forward primers are M hairpin barcode forward primers and/or the N reverse primers are N hairpin barcode reverse primers. Each hairpin barcode forward primer comprises, from the 5′ end to the 3′ end, a 5′ stem sequence, the adapter sequence, a unique molecular identifier (UMI), a 3′ stem sequence and the target-specific sequence complementary to a respective portion of a T-cell receptor (TCR) or B-cell receptor (BCR) clonotype. Each hairpin barcode reverse primer comprises, from the 5′ end to the 3′ end, a 5′ stem sequence, the adapter sequence, a UMI, a 3′ stem sequence and the target-specific sequence complementary to a respective portion of a TCR or BCR clonotype. At least a portion of the 5′ stem sequence of the hairpin barcode forward primer and/or the hairpin barcode reverse primer is complementary to at least a portion of the 3′ stem sequence of the hairpin barcode forward primer and/or the hairpin barcode reverse primer. The 5′ stem sequence and the 3′ stem sequence are configured to hybridize to each other at or under a closed annealing temperature and not hybridize to each other at or above an open annealing temperature. The method also comprises amplifying the nucleic acid molecules by performing polymerase chain reaction (PCR) pre-amplification of the nucleic acid molecules to form a plurality of barcoded PCR products. The PCR pre-amplification has an annealing temperature equal to or less than the closed annealing temperature of the hairpin barcode forward primers and/or the hairpin barcode reverse primers. The method further comprises contacting the plurality of barcoded PCR products with an adapter-specific forward primer and an adapter-specific reverse primer and amplifying the barcoded PCR products by performing PCR amplification on the barcoded PCR products to form a library of amplified barcoded PCR products. At least a portion of cycles of the PCR amplification has an annealing temperature equal to or greater than the open annealing temperature of the hairpin barcode forward primers and/or the hairpin barcode reverse primer. The method further comprises sequencing at least a respective portion of the amplified barcoded PCR products to form respective sequence reads comprising the UMI(s) and TCR or BCR sequence(s). The method also comprises demultiplexing the sequence reads based on nucleic acid sequences of the UMIs and mapping the demultiplexed sequence reads to respective TCR or BCR clonotypes based on nucleic acid sequences of the TCR or BCR sequences. The lymphocyte clonality is then determined for the sample based on the demultiplexed and mapped sequence reads.

Another aspect of the invention relates to a method of disease characterization. The method comprises determining lymphocyte clonality according to above of a sample comprising nucleic acid molecules of lymphocytes obtained from a subject. The method also comprises characterizing a disease of the subject based on the determined lymphocyte clonality. In an embodiment, the disease is selected from the group consisting of a hematologic disease, an infectious disease, a cancer disease and an autoimmune disease.

The present invention provides a very sensitive lymphocyte clonality determination that solves issues with non-uniform amplification of TCR and/or BCR clonotypes and polymerase-induced errors during sequencing. The invention can be applied to various sample types, including enriched and non-enriched cell populations. The method is simple to conduct, does not require any target nucleic acid molecule capture and provides quantitative information of TCR and/or BCR clonotypes.

### BRIEF DESCRIPTION OF THE DRAWINGS

The embodiments, together with further objects and advantages thereof, may best be understood by making reference to the following description taken together with the accompanying drawings, in which:

FIG. 1. Flow chart illustrating a method of determining lymphocyte clonality according to an embodiment.

FIG. 2. Schematic illustrations of primers (A, B, C) and amplification steps (A) used in a method of determining lymphocyte clonality according to an embodiment.

FIG. 3. Illustration of 6 TCR repertoire sequencing. (A) Schematic overview of the unrearranged TCR δ (TRD) locus, arrows show transcription direction. TRD variable 4 (TRDV4) to TRDV8 are shared between the TCR α (TRA) and TRD locus (see Table 1). (B) Amplification consists of two rounds of PCR. In the first barcoding PCR, target primers bind to the V and J genes, generating specific PCR products with UMIs and flanked adapter sequences. In the second adapter PCR, barcoded DNA is amplified with ILLUMINA® adapter primers. (C) The experimental workflow for VDJ-sequencing. (D) Design of synthetic reference molecules used for assay validation. The 32 gBlock molecules contain a TRDV segment consisting of the 97-163 last base pairs of the TRDV gene, and a 48-59 base pairs long TRD joining (TRDJ) segment containing the complete TRDJ gene. An internal unique template specific sequence for each primer pair combination is inserted between the TRDV and TRDJ segments. All synthetic gBlock molecules are also flanked by a non-amplified general sequence. All synthetic reference molecules are shown in Table 1.

FIG. 4. Performance of 32-plex assay targeting TRD sequences. (A) Dynamic range of 32-plex assay. Quantitative PCR on barcoded synthetic gBlock molecules, ranging from 2×10⁷ to 20 molecules per standard with 10-fold dilution steps. Cycle of quantification value (Cq-value) is shown, n=3. The amplification efficiency was calculated from the slope of the standard curve as 10^{1/(-slope)}−1. (B) Individual assay performance. The number of molecules quantified by sequencing using 2000, 200, and 20 synthetic gBlock molecules per assay. The standard curve of each assay is shown, n=3. The sequencing efficiency of each assay is shown in Table 4. (C) Assay sensitivity and reproducibility. Sequencing of two pools (A and B) of synthetic gBlock molecules with approximately 10 molecules for half of the assays and 1000 molecules for the other half. Synthetic gBlock molecules that were in minority in the first pool were in majority in the second pool. Mean±SD is shown, n=3.

FIG. 5. Immune repertoire sequencing of γδ T cells. (A) Number of productive TRD molecules for each assay, versus starting amount of γδ T-cell DNA. TRDJ assays are divided into separate panels and TRDV assays in different gray scales. The linear correlation of each assay is shown, n=3. (B) Visualization of the ten most productive clonotypes versus the amount of γδ T-cell DNA. Each gray scale is a single clonotype. Standard curves are shown, n=3. (C) Unique Molecule Identifier (UMI) distribution. The histogram shows how many times a particular UMI is sequenced, i.e., UMI family size. Data are generated from a representative γδ T-cell sample, containing 500 ng DNA. (D) Correction of molecule quantification using UMIs. Relative frequencies of clonotypes using raw sequencing reads (y-axis) versus using UMI (x-axis) are shown. Absolut molecule count based on UMI is shown on top x-axis. Data from a representative sample is show. (E) Sequencing reproducibility and sampling ambiguity. Coefficient of variation versus average number of barcode families detected is shown. Data are from 500 ng γδ T-cells DNA, n=3. (F) Distribution of molecules with different GC-content. (G) UMI family size distribution in relation to different GC-content. 1^{st} and 99^{th} percentile is at 46.0 and 54.4% GC content, respectively. (H) Distribution of molecules with different amplicon length. (I) UMI family size distribution in relation to amplicon length, 1^{st} and 99^{th} percentile is at 92 and 156 nucleotides, respectively. Raw sequencing reads were used in FIGS. 5C, 5F-5I.

FIG. 6. The immune repertoire of TRD in healthy individuals. (A) The number of productive TRD molecules detected by sequencing compared with the amount of γδ T-cell DNA used. (B) Treemapping of all clonotypes across ten healthy individuals. Each square represents a unique clonotype. The area of the square indicates the clonotype frequency and the gray scale shows which V and J genes was used. (C) Comparison of gene usage analyzed by FACS and immune repertoire sequencing (Seq). The usage of TRDV1 and TRDV2 as frequencies is shown. The Spearman's rank correlation coefficient is 0.88 (p<0.01) for TRDV1 and 0.94 (p<0.01) for TRDV2 when comparing FACS with sequencing.

FIG. 7. Comparison of immune repertoire sequencing of enriched and non-enriched γδ T cells from peripheral blood mononuclear cells (PBMCs). PBMCs from one individual were split in half: one sample was enriched for γδ T cells, while the other was analyzed directly without cell enrichment. (A) Each point is a unique clonotype, and its frequencies as non-enriched and enriched are shown. Line shows linear regression. Outliers are indicated with arrows. (B) Relative frequencies of the 20 most commonly detected clonotypes in the γδ T cell enriched and non-enriched samples, respectively. Amino acid sequence of the CRD3 region for each clonotype is shown (SEQ ID NO: 52 to 70). Non-overlapping (black) is the cumulative frequency of all clonotypes only detected in one of the samples. Non-shown (grey) is the cumulative frequency of all overlapping clones that are not among the top 20 clonotypes.

FIG. 8. Barcode primer validation. A five-step dilution curve from 10,000 to 16 synthetic gBlock molecules was used for assay validation. (A) Amplification curves of adapter PCR using different amounts of barcoded DNA. A representative assay is shown. (B) Melting curve analysis of the final libraries using the same assay as shown in (A). Arrows indicate specific (full arrow) and unspecific (hatched arrow) PCR products, respectively. (C) Fragment size analysis. Arrows indicate specific (full arrow) and non-specific (hatched arrow) PCR products. Electropherograms with 10,000, 80 and zero synthetic gBlock molecules as starting material are shown.

FIG. 9. Number of detected molecules versus number of starting synthetic gBlock molecules. Total number of molecules from the 32-plex assay, quantified using sequencing. The standard curve generated from three different amounts of loaded synthetic molecules is shown, n=3.

FIG. 10. Primer pair specificity. Synthetic gBlock DNA standards were used to test off-target amplification. Each TRDJ and TRDV primer was analyzed individually by selecting all assembled reads based on exact primer sequence. The number of UMI families detected containing the primer sequence (x-axis) was compared to the number of UMI families containing a template specific sequences (for details, see FIG. 3D and Table 1) associated with a TRDV or TRDJ gene (y-axis). (A) Shows TRDV primers with matching TRDV associated synthetic molecules. (B) Shows TRDJ primer with matching TRDJ associated synthetic molecule. Analysis was performed using the pooled assembled sequences from the three replicates of the 2000 synthetic molecule sample shown in FIG. 4.

FIG. 11. Quantification of synthetic gBlock molecule pools using quantitative PCR. Synthetic gBlock molecules that were in minority in the first pool were in majority in the second pool. Cycle of quantification value (Cq-value) is shown for 32 synthetic gBlock molecules. Mean±SD is shown, n=3.

FIG. 12. Clonotype size distribution in healthy donors. Histograms of the number of TRD clonotypes of various sizes across the ten healthy individuals.

FIG. 13. Treemapping showing the gene usage of all clones across ten healthy donors. Each square represents the use of a specific TRAV/TRDV—TRDJ gene combination, as defined by different gray scales. The area of each square indicates the accumulated frequency of all clones using a particular gene combination.

FIG. 14. Flow cytometry gating. Gating strategy used to define γδ T cells (CD3⁺CD19⁻TCRγδ⁺) for cell sorting and/or analysis. Lymphocytes were gated based on (A) Side SCatter Area (SSC-A) versus Forward SCatter Area (FSC-A) and singlets were identified from the (B) FSC-A versus FSC-Hight (FSC-H) plot. (C) Dead cells were excluded and (D) T cells were identified as CD3⁺CD19⁻. (E) γδ T cells were identified among the CD3⁺ cells as TCRγδ⁺ and (F) subdivided into Vδ1⁺, Vδ2⁺ (TRDV1 and TRDV2, respectively using IMGT nomenclature) and Vδ1⁻Vδ2⁻ populations. The pseudo gray scale plots are representative examples of PBMCs isolated from peripheral blood of healthy donor.

FIG. 15. Immune repertoire sequencing of the IGH locus for three B cells.

### DETAILED DESCRIPTION

The present embodiments generally relate to lymphocyte clonalities and, in particular to determining lymphocyte clonality.

The sequencing of immunoreceptor repertoires of B and T lymphocytes and tracking of specific clones provide essential information regarding many aspects of immunity, such as the extent of immune responses, localization of specific responding cells, the duration of immune responses, and presence of immune memory. The present invention provides an ultrasensitive immune repertoire sequencing approach that can be used to determine lymphocyte clonality, such as represented by T-cell receptor (TCR) and/or B-cell receptor (BCR) clonotypes in a sample.

There are several challenges when analyzing lymphocyte clonality in a sample including, for instance, determining the exact number and size of the TCR and/or BCR clonotypes, no prior information of the number of different TCR and/or BCR clonotypes in the sample, distinguishing between clonotypes with similar sequences from sequence errors introduced during amplification and/or sequencing.

As used herein, each T cell or B cell clone carries a unique clonotype defined by the nucleotide sequence that arises during the gene rearrangement process for the TCR or BCR. In most applications, the clonotype is regarded as being defined by the complementarity-determining region 3 (CDR3) of the TCR or BCR, such as the CDR3 of the alpha (α) chain of the TCR (TRA) for αβ TCRs and T cells, or more preferably the CDR3 of the beta (β) chain of the TCR (TRB) for αβ TCRs and T cells, or the CDR3 of the gamma (γ) chain of the TCR (TRG) for γδ TCRs and T cells, or more preferably the CDR3 of the delta (6) chain of the TCR (TRD) for γδ TCRs and T cells, or the CDR3 of the BCR for B cells.

The TCR alpha (α) chain is generated by recombination of variable (V) and joining (J) gene segments, whereas the TCR beta (β) chain is generated by recombination the V, diversity (D) and joining (J) gene segments. Correspondingly, the TCR gamma (γ) chain involves V and J recombination, whereas the TCR delta (δ) chain is generated by V, D and J recombination.

Determination of lymphocyte clonality could thereby be used to determine the particular combination of V (TRAV) and J (TRAJ) gene segments of the TCR alpha (α) chain and/or the particular combination of V (TRBV) and D (TRBD) gene segments, D and J (TRBJ) gene segments or V, D and J gene segments of the TCR beta (β) chain for αβ T cells. Correspondingly, determination of lymphocyte clonality could be used to determine the particular combination of V (TRGV) and J (TRGJ) gene segments of the TCR gamma (γ) chain and/or the particular combination of V (TRDV) and D (TRDD) gene segments, D and J (TRDJ) gene segments or V, D and J gene segments of the TCR delta (δ) chain for γδ T cells.

The BCR is also generated based on V, D and J recombination. This means that determination of lymphocyte clonality could be used to determine the particular combination of V and J gene segments, D and J gene segments or V, D and J gene segments of the BCR for B cells.

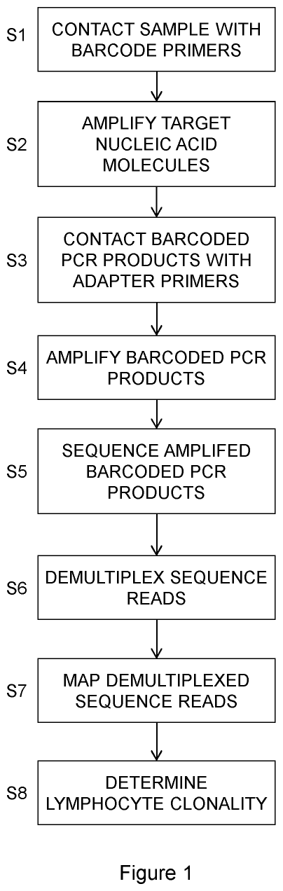

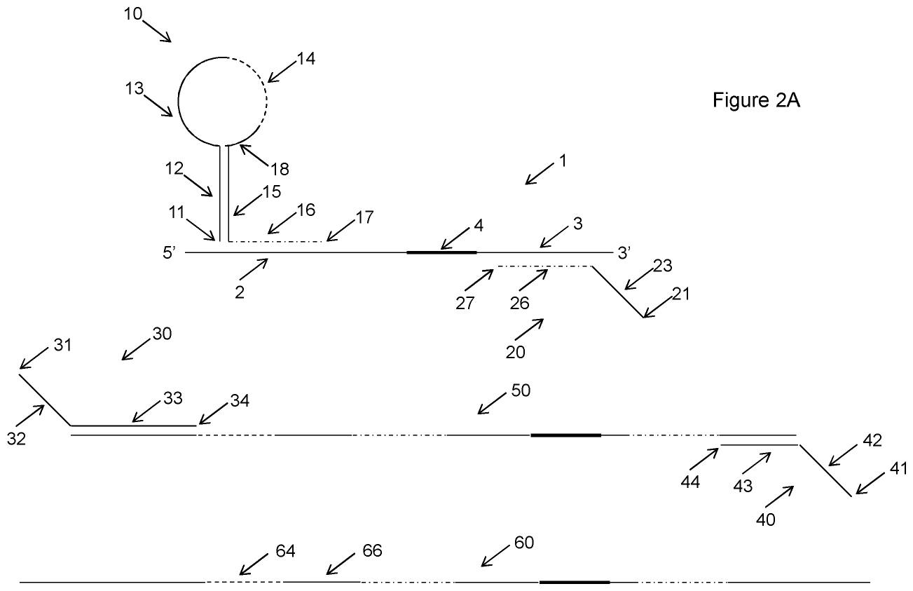

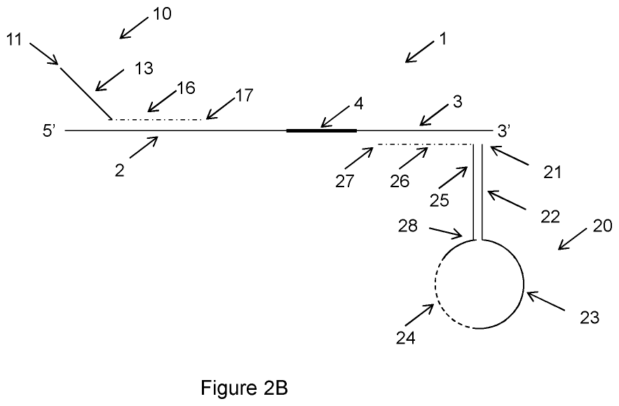

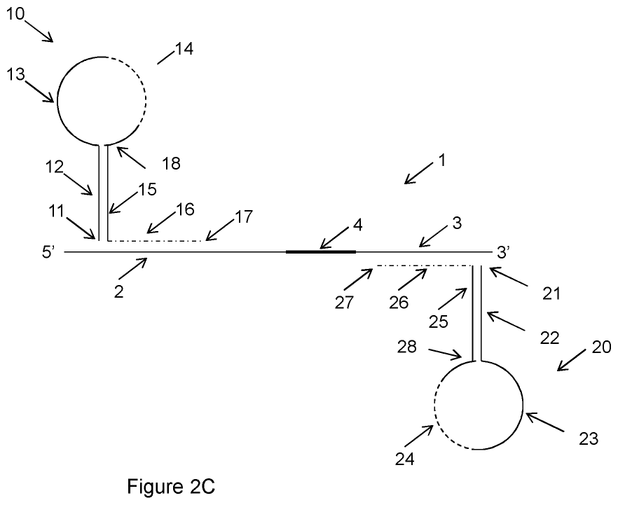

An aspect of the invention relates to a method of determining lymphocyte clonality, see FIGS. 1 and 2A to 2C. The method comprises contacting, in step S1, a sample comprising nucleic acid molecules 1 of lymphocytes with M forward primers 10 and N reverse primers 20.

In an embodiment, M is an integer equal to or larger than one and N is an integer equal to or larger than one. The M forward primers 10 comprise, from a 5′ end 11 to a 3′ end 17, an adapter sequence 13 and a target-specific sequence 16. Correspondingly, the N reverse primers 20 comprise, from a 5′ end 21 to a 3′ end 27, an adapter sequence 23 and a target-specific sequence 26.

In an embodiment, the M forward primers 10 are M hairpin barcode forward primers 10 and/or the N reverse primers 20 are N hairpin barcode reverse primers 20. In such an embodiment, each hairpin barcode forward primer 10 comprises, from the 5′ end 11 to the 3′ end 17, a 5′ stem sequence 12, the adapter sequence 13, a unique molecular identifier (UMI) 14, a 3′ stem sequence 15 and the target-specific sequence 16 complementary to a respective portion 2 of a TCR or BCR clonotype. Correspondingly, each hairpin barcode reverse primer 20 comprises, from the 5′ end 21 to the 3′ end 27, a 5′ stem sequence 22, the adapter sequence 23, a UMI 24, a 3′ stem sequence 25 and the target-specific sequence 26 complementary to a respective portion 3 of a TCR or BCR clonotype. At least a portion of the 5′ stem sequence 12, 22 of the hairpin barcode forward primer 10 and/or the hairpin barcode reverse primer 20 is complementary to at least a portion of the 3′ stem sequence 15, 25 of the hairpin barcode forward primer 10 and/or the hairpin barcode reverse primer 20. The 5′ stem sequence 12, 22 and the 3′ stem sequence 15, 25 are configured to hybridize to each other at or under a closed annealing temperature and not hybridize to each other at or above an open annealing temperature.

The method as shown in FIG. 1 also comprises amplifying, in step S2, the nucleic acid molecules 1 by performing polymerase chain reaction (PCR) pre-amplification of the nucleic acid molecules 1 to form a plurality of barcoded PCR products 50. The PCR pre-amplification as performed in step S2 has an annealing temperature equal to or less than the closed annealing temperature of the hairpin barcode forward primers 10 and/or the hairpin barcode reverse primers 20. The method further comprises contacting, in step S3, the plurality of barcoded PCR products 50 with an adapter-specific forward primer 30 and an adapter-specific reverse primer 40 and amplifying, in step S4, the barcoded PCR products 50 by performing PCR amplification on the barcoded PCR products 50 to form a library of amplified barcoded PCR products 60. In an embodiment, at least a portion of cycles of the PCR amplification has an annealing temperature greater than or equal to the open annealing temperature of the hairpin barcode forward primers 10 and/or the hairpin barcode reverse primer 20.

The method also comprises sequencing, in step S5, at least a respective portion of the amplified barcoded PCR products 60 to form respective sequence reads comprising the UMI(s) 64 and TCR or BCR sequence(s) 66. The method further comprises demultiplexing, in step S6, the sequence reads based on nucleic acid sequences of the UMIs 64 and mapping, in step S7, the demultiplexed sequence reads to respective TCR or BCR clonotypes based on nucleic acid sequences of the TCR or BCR sequences 66. The lymphocyte clonality for the sample is then determined in step S8 based on the demultiplexed and mapped sequence reads.

The sample used in the determination of lymphocyte clonality could be any sample comprising nucleic acid molecules 1 of lymphocytes, such as T cells or lymphocytes, B cells or lymphocytes or T and B cells or lymphocytes.

In an embodiment, the sample is an enriched lymphocyte sample, in which lymphocytes have been enriched and isolated from a biological sample, such as taken from a subject. For instance, lymphocytes could be enriched from buffy coats or peripheral blood mononuclear cells (PBMCs) using immunomagnetic cell separation, fluorescence-activated cell sorting (FACS) or other lymphocyte enriching techniques.

However, an advantage of the present invention is that no lymphocyte enrichment is needed in order to determine lymphocyte clonality. For instance, PBMCs could be isolated from buffy coat of a subject and used as sample in the method shown in FIG. 1.

The sample could therefore be a lymphocyte enriched sample or a biological sample that is not subject to lymphocyte enrichment. Examples of the latter include a blood sample or a buffy coat sample. The buffy coat is the fraction of an anticoagulated blood sample that contains most of the lymphocytes and platelets following density gradient centrifugation. In fact, the present invention can be used for any sample comprising lymphocytes or lymphocyte nucleic acid molecules, also including tissue samples, a fixed sample, i.e., a biological sample, such as a tissue sample, that has been subject to fixation to preserve the biological sample.

True clonal variation is difficult to separate from sequencing errors. By comparing the frequency of multiple clonotypes that differ from each other by merely one or a few nucleotides, many clonality determining methods that are based on bioinformatical processes assume that low-frequency clonotype(s) is(are) a sequencing mistake and instead interpret such a low-frequency clonotype as being one of the more frequently expressed clonotypes but with one or a few sequencing errors. Hence, a sample containing a low-frequency clonotype and a high-frequency clonotype may in such a case be misinterpreted to only contain the high-frequency clonotype.

Furthermore, amplification steps in the clonality determination introduce PCR-induced amplification biases and errors, which effectively prevent accurate clonality determination and quantification. UMIs are known to be used to reduce such PCR-induced amplification biases and errors. To date, UMIs have mostly been applied to messenger ribonucleic acid (mRNA) when profiling the immune receptor repertoire of B and T cells to remove PCR duplicates and improving sequence accuracy. However, the analysis of mRNA has several disadvantages. Firstly, the number of transcripts per cell is not constant. Hence, the correct number and size of various T or B cell clones cannot be accurately estimated. Secondly, the reverse transcription efficiency is variable between sequences, and reverse transcriptase is up to 1000 times more prone to introduce errors than deoxyribonucleic acid (DNA) polymerase, causing both quantification and sequence biases. Hence, the nucleic acid molecules 1 are DNA molecules 1 and the sample comprises DNA molecules 1 of lymphocytes.

UMIs can be added to DNA by either ligation- or PCR-based approaches. Ligation-based UMI approaches require that target DNA is captured before the analysis, otherwise, all genomic DNA will be analyzed. Another limitation is that molecules are lost due to limited ligation efficiency. In comparison, PCR-based UMI approaches are simpler since no capture step is needed. PCR-based methods are potentially also more sensitive since they do not suffer from ineffective capture and ligation steps. However, introduction of UMIs into PCR primers may cause massive formation of non-specific PCR products caused by the random nucleotide sequence of UMIs. This problem is solved according to the present invention by shielding the UMIs 14, 24 in secondary structures in the hairpin barcode forward primers 10 and/or the hairpin barcode reverse primers 20. Hence, in order to minimize the formation of non-specific PCR products, the UMI 14, 24 is protected inside a hairpin loop that opens and closes its secondary structure in a temperature-dependent manner.

Hairpin primers have been proposed in the art in connection with determination of allele variants, such as mutation analysis, see for instance U.S. Pat. No. 10,557,134. However, compared to such allele variant detection, immune repertoire sequencing and lymphocyte clonality determination are much more challenging since the sequences of individual target DNA molecules, i.e., TCR or BCR encoding DNA molecules, are different from each other including, having different length and GC-content, in addition to being present in different copy numbers, i.e., high frequency TCR or BCR clones vs. low frequency TCR or BCR clones. Allele variant analysis regularly only includes the detection of two different sequences, such as wild-type (wt) sequence and mutant sequence, often with a single nucleotide variant. Accordingly, it was highly surprising that lymphocyte clonality could be determined in a UMI-based approach using UMIs 14, 24 protected inside the hairpin barcode forward primers 10 and/or inside the hairpin barcode reverse primers 20 given the fundamental differences between lymphocyte clonality determination and allele variant determination. Hence, the priming strategy of the present invention with UMI 14, 24 protected inside hairpin loops of the hairpin barcode forward primers and/or hairpin barcode reverse primers 20 enabled accurate lymphocyte determination, including quantification, with specific nucleotide resolution even though the amplified nucleic acid sequences, i.e., barcoded PCR products 50 and amplified barcoded PCR products 60, are different in sequence content and length.

In an embodiment, the forwards primers 10 are hairpin barcode forward primers 10, whereas the reverse primers 20 do not form any hairpin or loop structure as shown in FIG. 2A. In such an embodiment, step S1 comprises contacting the sample comprising nucleic acid molecules 1 of lymphocytes with a set of hairpin barcode forward primers 10 and at least one reverse primer 20. Each hairpin barcode forward primer 10 of the set comprises, from the 5′ end 11 to the 3′ end 17, the 5′ stem sequence 12, the adapter sequence 13, the UMI 14, the 3′ stem sequence 15 and the target-specific sequence 16 complementary to a respective portion 2 of a TCR or BCR clonotype. The at least one reverse primer 20 comprises, from the 5′ end 21 to the 3′ end 27, the adapter sequence 23 and the target-specific sequence 23.

In an embodiment, all hairpin barcode forward primers 10 comprise the same 5′ stem sequence 12, the same adapter sequence 13 and/or the same 3′ stem sequence 15. However, the hairpin barcode forward primers 10 comprise different UMIs 14. The set may comprise hairpin barcode forward primers all having the same target-specific sequence 16 (but different UMIs), multiple, i.e., at least two, hairpin barcode forward primers 10 having the same target-specific sequence 16 or the hairpin barcode forward primers 10 in the set may have different target-specific sequences 16.

An illustrative, but non-limiting, example of hairpin barcode forward primers 10 that can be used in accordance with the embodiments have the following sequence (SEQ ID NO: 13):

5′-
GGACACTCTTTCCCTACACGACGCTCTTCCGATCTNNNNNNNNNNNNAT
GGGAAAGAGTGTCC-target-specific sequence-3′

In this illustrative example, the 5′ stem sequence 12 comprises, preferably consists of, the nucleotide sequence GGACACTCTTTCCC (SEQ ID NO: 48) that is complementary to and capable of hybridizing to the 3′ stem sequence 15 comprising, preferably consisting of, the nucleotide sequence GGGAAAGAGTGTCC (SEQ ID NO: 49). In this example, NNNNNNNNNNNN represents the UMI 14. The hairpin barcode forward primer 10 additionally comprises a nucleotide sequence forming the adapter sequence 13. This adapter sequence 13 may be positioned between the 5′ stem sequence 12 and the UMI 14. It is, however, also possible that all or a 3′ portion of the nucleotides of the 5′ stem sequence 12 also form part of the adapter sequence 13. For instance, an adapter sequence 14 of ACACTCTTTCCCTACACGACGCTCTTCCGATCT (SEQ ID NO: 50) could be used in the above illustrated example.

In an embodiment, a single or common reverse primer 20 is used together with the hairpin barcode forward primers 10. The common reverse primer 20 comprises a target-specific sequence 23 that is complementary to sequence or region 3 of the nucleic acid molecule 1 that is common for the different TCR or BCR clonotypes.

In another embodiment, multiple different reverse primers 20 is used together with the hairpin barcode forward primers 10. Each reverse primer 20 then preferably comprises a respective target-specific sequence 23 that is complementary to a respective sequence or region 3 of the nucleic acid molecule 1 that is specific for a given TCR or BCR clonotype or specific for a given group of TCR or BCR clonotypes, i.e., is not common for all different TCR or BCR clonotypes.

In an embodiment, all reverse primers 20 comprise the same adapter sequence 23. However, the reverse primers 20 may comprise different target-specific sequences 26.

An illustrative, but non-limiting, example of reverse primers 20 that can be used in accordance with the embodiments have the following sequence (SEQ ID NO: 14):

5′-GTGACTGGAGTTCAGACGTGTGCTCTTCCGATCT-
target-specific region-3′

In an embodiment, the adapter sequence 13 of the hairpin barcode forward primers 10 is different from the adapter sequence 23 of the at least one reverse primer 20.

In another embodiment, the reverse primers 20 are hairpin barcode reverse primers 20, whereas the forward primers 10 do not form any hairpin or loop structure as shown in FIG. 2B. In such an embodiment, step S1 comprises contacting the sample comprising nucleic acid molecules 1 of lymphocytes with a set of hairpin barcode reverse primers 20 and at least one forward primer 10. Each hairpin barcode reverse primer 20 of the set comprises, from the 5′ end 21 to the 3′ end 27, the 5′ stem sequence 22, the adapter sequence 23, the UMI 24, the 3′ stem sequence 25 and the target-specific sequence 26 complementary to a respective portion 3 of a TCR or BCR clonotype. The at least one forward primer 10 comprises, from the 5′ end 11 to the 3′ end 17, the adapter sequence 13 and the target-specific sequence 13.

In an embodiment, all hairpin barcode reverse primers 20 comprise the same 5′ stem sequence 22, the same adapter sequence 23 and/or the same 3′ stem sequence 25. However, the hairpin barcode reverse primers 20 comprise different UMIs 24. The set may comprise hairpin barcode reverse primers all having the same target-specific sequence 26 (but different UMIs), multiple hairpin barcode reverse primers 20 having the same target-specific sequence 26 or the hairpin barcode reverse primers 20 in the set may have different target-specific sequences 26.

In an embodiment, a single or common forward primer 10 is used together with the hairpin barcode reverse primers 20. The common forward primer 10 comprises target-specific sequence 13 that is complementary to sequence or region 2 of the nucleic acid molecule 1 that is common for the different TCR or BCR clonotypes.

In another embodiment, multiple different forward primers 10 is used together with the hairpin barcode reverse primers 20. Each forward primer 10 then preferably comprises a respective target-specific sequence 13 that is complementary to a respective sequence or region 2 of the nucleic acid molecule 1 that is specific for a given TCR or BCR clonotype or specific for a given group of TCR or BCR clonotypes, i.e., is not common for all different TCR or BCR clonotypes.

In an embodiment, all forward primers 10 comprise the same adapter sequence 13. However, the forward primers 10 may comprise different target-specific sequences 16.

In an embodiment, the adapter sequence 23 of the hairpin barcode reverse primers 20 is different from the adapter sequence 13 of the at least one forward primer 10.

In a further embodiment, both the forward primers 10 and the reverse primers 20 are hairpin barcoded primers as shown in FIG. 2C. In such an embodiment, step S1 comprises contacting the sample comprising nucleic acid molecules 1 of lymphocytes with a first set of hairpin barcode forward primers and a second set of hairpin barcode reverse primers 20. Each hairpin barcode forward primer 10 of the first set comprises, from the 5′ end 11 to the 3′ end 17, the 5′ stem sequence 12, the adapter sequence 13, a first UMI 14, the 3′ stem sequence 15 and the target-specific sequence 16 complementary to a respective portion 2 of a TCR or BCR clonotype and each hairpin barcode reverse primer 20 of the second set comprises, from the 5′ end 21 to the 3′ end 27, the 5′ stem sequence 22, the adapter sequence 23, a second UMI 24, the 3′ stem sequence 25 and the target-specific sequence 26 complementary to a respective portion 3 of a TCR or BCR clonotype.

In an embodiment, all hairpin barcode forward primers 10 of the first set comprise the same 5′ stem sequence 12, the same adapter sequence 13 and/or the same 3′ stem sequence 15. However, the hairpin barcode forward primers 10 comprise different UMIs 14.

In an embodiment, all hairpin barcode reverse primers 20 of the second set comprise the same 5′ stem sequence 22, the same adapter sequence 23 and/or the same 3′ stem sequence 25. However, the hairpin barcode reverse primers 20 comprise different UMIs 24.

In an embodiment, the 5′ stem sequence 12 of the hairpin barcode forward primers 10 is the same as the 5′ stem sequence 22 of the hairpin barcode reverse primers 20. In another embodiment, the 5′ stem sequence 12 of the hairpin barcode forward primers 10 is different from the 5′ stem sequence 22 of the hairpin barcode reverse primers 20. Correspondingly, the 3′ stem sequence 15 of the hairpin barcode forward primers 10 could be the same as or different from the 3′ stem sequence 25 of the hairpin barcode reverse primers 20. However, the adapter sequence 13 of the hairpin barcode forward primers is preferably different from the adapter sequence 23 of the hairpin barcode reverse primers 20.

The hairpin barcode forward primers 10 and/or the hairpin barcode reverse primers 20 form a hairpin structure shielding the UMI 14, 24 inside the hairpin loop as shown in FIGS. 2A to 2C at a temperature equal to or below the closed annealing temperature. Hence, equal to or below this closed annealing temperature at least a portion of the 5′ stem sequence 12, 22 hybridizes to at least a portion of the 3′ stem sequence 15, 25. Correspondingly, when the temperature increases to or above the open annealing temperature, the least a portion of the 5′ stem sequence 12, 22 no longer hybridizes to the at least a portion of the 3′ stem sequence 15, 25.

The length of the 5′ stem sequence 12, 22 and the 3′ stem sequence 15, 25 of the hairpin barcode forward primers 10 and/or the hairpin barcode reverse primers 20, or rather the respective portions of these stem sequences 12, 22, 15, 25 that are configured to hybridize to each other define at least partly the closed and open annealing temperatures. Generally, the longer the hybridizing portions of the 5′ stem sequences 12, 22 and the 3′ stem sequences 15, 25, i.e., the higher number of nucleotides in these hybridizing portions, the higher the closed and open annealing temperatures. The closed and open annealing temperatures are not only defined based on the length of the hybridizing portions but also by the particular combination of nucleotides in the hybridizing portions. For instance, hybridizing portions having a higher GC content (G-C and C-G base pairing) have generally a higher temperature of melting (Tm) and thereby a higher closed and opened annealing temperature as compared to hybridizing portions of the same length but lower GC content, i.e., a higher AT content (A-T and T-A base pairing). Also modified nucleotides and nucleic acid analogues, for instance, peptide nucleic acid (PNA), locked nucleic acid (LNA), glycol nucleic acid (GNA), threose nucleic acid (TNA) and Zip nucleic acid (ZNA), present in the 5′ stem sequence 12, 22 and/or the 3′ stem sequence 15, 25 could be used to modify the closed and open annealing temperature. Such modified nucleotides and nucleic acid analogues could then be used to define and tailor the closed and open annealing temperatures of the hairpin barcode forward primers 10 and/or the hairpin barcode reverse primers 20. For instance, LNA generally increases the Tm of two complementary sequences as compared to the naturally occurring nucleotides A, T, G and C. Hence, introducing one or more of LNAs in the 5′ stem sequence 12, 22 and/or the 3′ stem sequence 15, 25 could be used to achieve a target closed or open annealing temperature but using shorter 5′ stem sequences 12, 22 and shorter 3′ stem sequences 15, 25 as compared to when using only naturally occurring nucleotides.

In a particular embodiment, the 3′ stem sequence 15, 25 comprises 5-15 nucleotides, preferably 8-15 nucleotides, and more preferably 12-15 nucleotides. Correspondingly, in a particular embodiment, the 5′ stem sequence 12, 22 comprises 5-15 nucleotides, preferably 8-15 nucleotides, and more preferably 12-15 nucleotides.

In an embodiment, the hairpin barcode forward primer 10 and/or the hairpin barcode reverse primer 20 further comprises at least one destabilizing nucleotide 18, 28 between the UMI 14, 24 and the 3′ stem sequence 15, 25. In a particular embodiment, the hairpin barcode forward primer 10 and/or the hairpin barcode reverse primer 20 comprises at least two destabilizing nucleotides 18, 28, between the UMI 14, 24 and the 3′ stem sequence 15, 25. Such destabilizing nucleotide(s) can be incorporated to ensure that the UMI 14, 24 itself does not, by random chance, complement the adapter sequence 13, 23 and results in a longer, more stable stem having higher closed and open annealing temperatures.

In an embodiment, the UMI 14, 24 is a random n₁n₂n₃ . . . nₖ sequence. In this embodiment, nᵢ, i=1 . . . k, is one of A, T, C and G, and k is preferably from 6 up to 18, more preferably from 10 up to 15, and such as 12. Hence, each hairpin barcode primer 10 and/or hairpin barcode reverse primer 20 preferably comprises a respective unique UMI 14, 24 having a random sequence that is different from the random sequences of UMIs 14, 24 in other hairpin barcode primers 10 and/or hairpin barcode reverse primers 20. The length of the UMIs 14, 24 is preferably selected at least partly based on the number of nucleic acid molecules 1 in the sample. For instance, the number of unique UMIs 14, 24 is equal for 4^{k} for an UMI 14, 24 of length k nucleotides. This number 4^{k} should preferably be significantly larger than the number of nucleic acid molecules 1 in the sample.

The amplification of the nucleic acid molecule in step S2 comprises performing PCR pre-amplification at an annealing temperature equal to or less than the closed annealing temperature of the hairpin barcode forward primers 10 and/or the hairpin barcode reverse primers 20. Accordingly, at this PCR pre-amplification in step S2 at least a majority of the hairpin barcode forward primers 10 and/or hairpin barcode reverse primers 20 have an intact hairpin loop, i.e., the 5′ stem sequence 12, 22 is hybridized to the 3′ stem sequence 15, 25. This in turn significantly reduces the amount of non-specific PCR products that may otherwise occur during the pre-amplification by the random nucleotide sequence of the UMI 14, 24. Hence, the vast majority of the PCR products from the PCR-amplification in step S1 are the desired barcoded PCR products 50 corresponding to an amplified portion 2, 3, 4 of the nucleic acid molecule 1.

In an embodiment, the closed annealing temperature is equal to or less than 65° C. Hence, in such an embodiment, the PCR pre-amplification of step S2 is preferably performed at an annealing temperature equal to or less than to 65° C. For instance, the PCR pre-amplification of step S2 could be performed at an annealing temperature selected within an interval of from 60° C. up to 65° C., preferably from 60° C. up to 64° C., such as at about 62° C. The PCR pre-amplification of the nucleic acid molecule 1 is performed using a polymerase, preferably a DNA polymerase, and more preferably a heat-stable DNA polymerase. Non-limiting, but illustrative, examples of DNA polymerases that can be used according to the embodiments include Thermus thermophilus (Tth) DNA polymerase, Bacillus stearothermophilus DNA polymerase, Thermus aquaticus (Taq) DNA polymerase, Thermus flavus (Tfl) polymerase, Vent® DNA polymerase, Pfu polymerase, and Escherichia coli DNA polymerase I. In some embodiments, the DNA polymerase lacks 5′-nuclease activity. Examples of such polymerases include Klenow fragment of DNA polymerase 1, Stoeffel fragment of Taq polymerase, Pfu polymerase or Vent® polymerase. In an embodiment, the DNA polymerase is a so-called thermoactivated DNA polymerase, also referred to as, hot-start DNA polymerase. Specific examples of DNA polymerases include Takara PRIME STAR GXL polymerase I, Clontech's ADVANTAGE HD Polymerase, NEB Q5© High-Fidelity DNA Polymerases NEB PHUSION® High-Fidelity DNA Polymerases, ThermoFisher PLATINUM® Taq DNA Polymerase High Fidelity, ThermoFisher ACCUPRIME™ Pfx DNA Polymerase, ThermoFisher ACCUPRIME™ Taq DNA Polymerase High Fidelity, ThermoFisher Phusion™ High Fidelity Polymerase, ThermoFisher Platinum™ SuperFi™ II DNA Polhymerase, Promega Pfu DNA Polymerase, and Qiagen HOTSTAR HIFIDELITY Polymerase. The above presented examples of DNA polymerases that can be used in the PCR pre-amplification in step S2 may also be used in the PCR amplification in step S4.

In an embodiment, step S2 in FIG. 1 comprises amplifying the nucleic acid molecules 1 by performing 1-25 cycles of PCR pre-amplification of the nucleic acid molecules 1 to form the plurality of barcoded PCR products 50. In preferred embodiments, step S2 comprises amplifying the nucleic acid molecules 1 by performing 2-20 cycles and more preferably 2-15 cycles of PCR pre-amplification of the nucleic acid molecules 1 to form the plurality of barcoded PCR products 50. Hence, it is generally preferred to perform a rather low number of PCR cycles in the PCR pre-amplification in step S2. This low number of PCR cycles, together with protecting UMIs 14, 24 within hairpin loops, significantly reduces the amount of non-specific PCR products produced in step S2.

The barcoded PCR products 50 obtained in the amplification in step S2 are then contacted in step S3 with an adapter-specific forward primer 30 and an adapter-specific reverse primer 40 as indicated in FIG. 2A.

In an embodiment, the adapter-specific forward primer 30 comprises a sequence equal to or complementary to, preferably equal to, the adapter sequence 13 of the forward primers 10, such as of the hairpin barcode forward primers 10. Correspondingly, the adapter-specific reverse primer 40 comprises a sequence equal to or complementary to, preferably equal to, the adapter sequence 23 of the reverse primers 20.

In a particular embodiment, the adapter-specific forward primer 30 comprises, from a 5′ end 31 to a 3′ end 34, one of a P5 sequence and a P7 sequence 32 and the sequence 33 equal to or complementary to, preferably equal to, the adapter sequence 13 of the forward primers 10. In this particular embodiment, the adapter-specific reverse primer 40 comprises, from a 5′ end 41 to a 3′ end 44, the other of the P5 sequence and the P7 sequence 42 and the sequence 43 equal to or complementary to, preferably equal to, the adapter sequence 23 of the reverse primers 20. In a particular embodiment, the adapter-specific reverse primer 40 comprises, from the 5′ end 41 to the 3′ end 44, the other of the P5 sequence and the P7 sequence 42, an index sequence and the sequence 43 equal to or complementary to, preferably equal to, the adapter sequence 23 of the reverse primers 20.

In an embodiment, the P5 and P7 sequences are P5 and P7 ILLUMINA© sequences. In an embodiment, the P5 sequence comprises AATGATACGGCGACCACCGA (SEQ ID NO: 15). For instance, the P5 sequence could comprise, such as consist of, AATGATACGGCGACCACCGAGATCTACAC (SEQ ID NO: 51). In this latter example, at least one of the 3′ nucleotides of the P5 sequence may be common for the P5 sequence and the following sequence 33. Correspondingly, in an embodiment, the P7 comprises, preferably consists of, CAAGCAGAAGACGGCATACGAGAT (SEQ ID NO: 16).

The PCR amplification in step S4 is performed at an annealing temperature equal to or greater than the open annealing temperature of the hairpin barcode forward primers 10 and/or the hairpin barcode reverse primers 20. At such an annealing temperature, at least a significant portion of the barcoded PCR products 50 are in an open structure, i.e., there is no significant hairpin loop formation by hybridization of complementary stem portions of the barcoded PCR products 50. In a particular embodiment, the open annealing temperature is at least 70° C. In such a particular embodiment, the PCR amplification in step S4 is performed at a temperature equal to or above 70° C., such as 71° C., 72° C. or even higher.

In an embodiment, the open annealing temperature of the hairpin barcode forward primers 10 and/or the hairpin barcode reverse primers 20 is higher than the closed annealing temperature of the hairpin barcode forward primers 10 and/or the hairpin barcode reverse primers 20. In a particular embodiment, the open annealing temperature is at least 1° C., preferably at least 2° C., such at least 3° C. or 4° C., and more preferably at least 5° C. higher than the closed annealing temperature.

In an embodiment, step S4 in FIG. 1 comprises amplifying the barcoded PCR products 50 by performing at least 2 cycles of PCR amplification on the barcoded PCR products 50 to form a library of amplified barcoded PCR products 60. In preferred embodiments, step S4 comprises amplifying the barcoded PCR products 50 by performing at least 3 cycles, such as at least 5 cycles, and more preferably at least 10 cycles of PCR amplification on the barcoded PCR products 50 to form the library of amplified barcoded PCR products 60. For instance, at least 15 cycles, such as at least 20 cycles, such as at least 25 cycles or at least 30 cycles of PCR amplification are performed on the barcoded PCR products 50 to form the library of amplified barcoded PCR products 60. The number of PCR cycles is preferably selected to achieve sufficient number of amplified barcoded PCR products 60 for sequencing even for nucleic acid molecules 1 present in a low copy number in the sample.

In a general embodiment, the number of PCR cycles in the amplification in step S4 is higher than the number of PCR cycles in the amplification in step S2.

In an optional, but preferred embodiment, the method comprises an additional step following step S2 and prior to step S4 in FIG. 1. This additional step then comprises degrading the polymerase used for amplifying the nucleic acid molecules 1 in the PCR pre-amplification (in step S2) prior to amplifying the barcoded PCR products in the PCR amplification (in step S4).

Various techniques could be used to degrade polymerases including, but not limited to, heat treatment, chemical treatment, addition of protease inhibitors, sample dilution and enzymatic treatment. For instance, a protease could be added to the amplification products from step S2 to enzymatically degrade the polymerase used for amplifying the nucleic acid molecules 1 in the PCR pre-amplification. Such a degradation of the polymerase once the PCR pre-amplification has been completed additionally inhibits formation of non-specific PCR products.

In an optional embodiment, the library of amplified barcoded PCR products 60 obtained in step S4 is first cleaned up prior to sequencing in step S5. For instance, the library may be purified to remove contaminants, such as dNTPs, salts, primers, etc., that may otherwise interfere with the sequencing. An example of such a purification is to use beads, such as magnetic beads, to bind the amplified barcoded PCR products 60 and thereby remove the amplified barcoded PCR products 60 from the remaining reagents from the PCR amplification in step S4. An illustrative, but non-limiting, example of a purification kit that could be used is AMPure XP for PCR Purification.

The amplified barcoded PCR products 60 are then sequenced in step S5 of FIG. 1. The sequencing is achieved by means of at least one sequencing primer. The result of the sequencing in step S5 is respective sequence reads comprising at least nucleotide sequences of the UMI 64 and TCR or BCR sequence(s) 66.

In a particular embodiment, step S5 comprises in situ sequencing the at least a portion of the amplified barcoded PCR products 60 immobilized onto a solid support. For instance, the preferred P5 and P7 sequences introduced into the amplified barcoded PCR products 60 by means of the adapter-specific forward primers 30 and the adapter-specific reverse primers 40 could be used to immobilize the amplified barcoded PCR products 60 onto the solid support. In such an embodiment, the solid support preferably comprises immobilized nucleotide sequences complementary to the P5 sequence and/or immobilized nucleotide sequences complementary to the P7 sequence.

The in situ sequencing of step 56 preferably comprises in situ sequencing by synthesis of the at least a portion of the amplified product 60.

For instance, if the adapter-specific forward and reverse primers 30, 40 comprise P5 and P7 sequences, respectively, the ILLUMINA® sequencing technology could be used to in situ sequence at least a portion of the amplified barcoded PCR products 60 by synthesis. In more detail, the amplified barcoded PCR products 60 are immobilized on a flow cell surface designed to present the amplified barcoded PCR sequences 60 in a manner that facilitates access to enzymes while ensuring high stability of surface bound amplified barcoded PCR products 60 and low non-specific binding of fluorescently labeled nucleotides.

Sequence By Synthesis (SBS) uses two or four fluorescently labeled nucleotides to sequence the amplified barcoded PCR products 60 on the flow cell surface in parallel. During each sequencing cycle, a single labeled deoxynucleoside triphosphate (dNTP) is added to the nucleic acid chain. The nucleotide label serves as a terminator for polymerization so after each dNTP incorporation, the fluorescent dye is imaged to identify the base and then enzymatically cleaved to allow incorporation of the next nucleotide.

As an illustrative, but non-limiting, example ILLUMINA© MiniSeq system could be used to sequence the amplified barcoded PCR products 60.

Accurate quantification and sequence error correction with UMIs is predicated on a one-to-one relationship between the number of unique UMI barcodes at a given genomic locus and the number of unique sequence reads that have been sequenced. However, errors within the UMI sequence, including nucleotide substitutions during amplification and nucleotide miscalling and insertions or deletions (indels) during sequencing, create additional artefactual UMIs.

There are different bioinformatics models and methods for determining whether UMIs have the same nucleotide sequence or are regarded as being different UMIs, e.g., UMI error resolving methods (Smith et al. (2017)). The simplest method is to merge all UMIs, retaining only the UMI with the highest counts. For this method, the number of networks formed at a given locus is equivalent to the estimated number of unique molecules. This method is often referred to as cluster method. This method is expected to underestimate the number of unique molecules, especially for complex networks. A more accurate method is denoted adjacency method (Smith et al. (2017)), which attempts to correctly resolve the complex networks by using node counts. The most abundant node and all nodes connected to it are removed from the network. If this does not account for all the nodes in the network, the next most abundant node and its neighbors are also removed. This is repeated until all nodes in the network are accounted for. In the method, the total number of steps to resolve the network(s) formed at a given locus is equivalent to the number of estimated unique molecules. This method allows a complex network to originate from more than one UMI, although UMIs with an edit distance of two will always be removed in separate steps. The excess of UMIs pairs with an edit distance of two observed in the data sets indicate that some of these UMIs are artefactual. Reasoning that counts for UMIs generated by a single sequencing error should be higher than those generated by two errors and UMIs resulting from errors during the PCR amplification stage should have higher counts than UMIs resulting from sequencing errors, the directional method was developed (Smith et al. (2017)). Networks were generated from the UMIs at a single locus, in which directional edges connect nodes a single edit distance apart when nₐ≥2n_{b}−1, where nₐ and n_{b} are the counts of node a and node b. The entire directional network is then considered to have originated from the node with the highest counts. The ratio between the final counts for the true UMI and the erroneous UMI generated from a PCR error is dependent upon which PCR cycle the error occurs and the relative amplification biases for the two UMIs, but should rarely be less than twofold. This method allows UMIs separated by edit distances greater than one to be merged so long as the intermediate UMI is also observed, and with each sequential base change from the most abundant UMI, the count decreases. For this method, the number of directional networks formed is equivalent to the estimated number of unique molecules.

The sequence reads as obtained in step S5 are then demultiplexed based on the nucleic acid sequences of the UMIs 64 in step S6. In an embodiment, this step S6 comprises dividing the sequence reads into groups having a same nucleotide sequence of the UMIs 64, optionally with at most a predefined number of mismatches allowed for nucleotide sequences of UMIs 64 in a same group.

In a particular embodiment, this predefined number n is zero, one or two, and preferably zero or one.

Thus, the sequence reads are preferably divided into different groups based on the nucleotide sequences of the UMIs so that sequence reads having the same UMI sequence are included in the same group. In this grouping or division of sequence reads, it is possible, in an embodiment, to accept up to two nucleotide mismatches, preferably up to one mismatch, between UMI sequences that belong to the same group.

The demultiplexed sequence reads are then mapped in step S7 to respective TCR or BCR clonotypes based on nucleic acid sequences of the TCR or BCR sequences 66 in the sequence reads. In an embodiment, this step S7 comprises dividing demultiplexed sequence reads into groups having a same nucleotide sequence of the TCR or BCR sequences 66, optionally with at most a predefined number of mismatches allowed for nucleotide sequences of TCR or BCR sequences 66 in a same group.

In a particular embodiment, the predefined number m is an integer equal to or larger than 0 but no larger than ten, preferably no larger than nine, such as no larger than eight or seven, or even no larger than six or five. Thus, within each group of sequence reads following the demultiplexing in step S6, the sequence reads are further divided into groups, or sub-groups, based on the nucleotide sequences of the TCR or BCR sequences 66. This means that sequence reads having the same TCR or BCR sequences 66 are grouped into the same group. In this grouping or division of sequence reads, it is possible, in an embodiment, to accept up to 10 nucleotide mismatches, preferably up to 5-6 nucleotide mismatches, between TCR or BCR sequences 66 that belong to the same group. Mismatches are allowed in order to accept possible polymerase-caused nucleotide substitutions during library preparation and sequencing procedure.

The lymphocyte clonality is then determined for the sample based on the demultiplexed and mapped sequence reads in step S8. In an embodiment, this step S8 comprises identifying any TCR or BCR clonotypes present in the sample. Hence, this step S8 preferably comprises identifying all TCR or BCR clonotypes that are present in the sample to thereby identify individual T cell or B cell clones in the sample. In an embodiment, step S8 also, or additionally, comprises quantifying the TCR or BCR clonotypes present in the sample based on the demultiplexed and mapped sequence reads.

Hence, not only the identities of the different TCR or BCR clonotypes in the sample are determined in step S8 but also a quantity thereof. Such a quantification of TCR or BCR clonotypes is possible due the presence of UMI 14, 24 in the hairpin barcode forward primers 10 and/or the hairpin barcode reverse primers 20. The quantification can be an absolute quantification, i.e., the total copy number of each TCR or BCR clonotype present in the sample, or a relative quantification, e.g., frequencies of the different TCR or BCR clonotypes present in the sample such as relative to each other or a reference sequence present in or added to the sample or the reaction mixtures in any of the steps S2 to S4.

In a particular embodiment, quantifying the TCR or BCR clonotypes comprises quantifying TCR or BCR clonotypes based on determining the number of different UMIs 64, optionally with at most a first predefined number mismatches, having the same nucleotide sequence of the TCR or BCR sequence 66, optionally with at most a second predefined number of mismatches.

In a particular embodiment, the first predefined number n is zero, one or two, preferably zero or one, and the second predefined number m is an integer equal to or larger than 0 but no larger than ten, preferably no larger than eight and more preferably no larger than five.

For instance, the Molecular Identifier Guided Error Correction (MIGEC) pipeline could be used in the demultiplexing and mapping steps S6 and S7. MIGEC comprises de-multiplexing, adapter trimming and read overlapping for sequence reads data and extraction of UMI sequences. MIGEC also comprises assembly of consensuses for original molecules that entered library preparation by grouping reads with identical molecular identifiers and mapping of V, D and J segments, extraction of CDR3 regions and clonotype assembly for all human and mouse immune receptors (TRA/TRB/TRG/TRD and IGH/IGL/IGK). Further features of MIGEC include additional filtering of hot-spot errors in CDR3 sequence.

In an embodiment, if M is one then N is equal to or larger than two and if N is one then M is equal to or larger than two. In this particular embodiment, multiple different forward primer 10 and/or multiple different reverse primers 20 are used in the method.

In an embodiment, determining lymphocyte clonality comprises determining clonality of αβ TCRs and αβ T cells. In a particular embodiment, the M forward primers 10 comprise at least one forward primer per V region of a TCR alpha (α) chain and the N reverse primers 20 comprise at least one reverse primer 20 per J region of the TCR alpha (α) chain. In other particular embodiments, the M forward primers 10 comprise at least one forward primer 10 per V region of a TCR beta (β) chain and the N reverse primers 20 comprise at least one reverse primer 20 per D region of the TCR beta (β) chain, or the M forward primers 10 comprise at least one forward primer 10 per D region of the TCR beta (β) chain and the N reverse primers 20 comprise at least one reverse primer 20 per J region of the TCR beta (β) chain, or the M forward primers 10 comprise at least one forward primer 10 per V region of the TCR beta (β) chain and the N reverse primers 20 comprise at least one reverse primer 20 per J region of the TCR beta (β) chain.

It is also possible to determine clonality of both the TCR alpha (α) chain and the TCR beta (β) chain in the same sample. In such a case, combinations of forward and reverse primers 10, 20 according to above are used in the same reaction mixture, or the sample is split into two samples and the two clonality determinations are run in parallel.

In another embodiment, determining lymphocyte clonality comprises determining clonality of γδ TCRs and γδ T cells. In a particular embodiment, the M forward primers 10 comprise at least one forward primer 10 per V region of a TCR gamma (γ) chain and the N reverse primers 20 comprise at least one reverse primer 20 per J region of the TCR gamma (γ) chain. In other particular embodiments, the M forward primers 10 comprise at least one forward primer 10 per V region of a TCR delta (δ) chain and the N reverse primers 20 comprise at least one reverse primer 20 per D region of the TCR delta (δ) chain, or the M forward primers 10 comprise at least one forward primer 10 per D region of the TCR delta (δ) chain and the N reverse primers 20 comprise at least one reverse primer 20 per J region of the TCR delta (δ) chain, or the M forward primers 10 comprise at least one forward primer 10 per V region of the TCR delta (δ) chain and the N reverse primers 20 comprise at least one reverse primer 20 per J region of the TCR delta (δ) chain.

It is also possible to determine clonality of both the TCR gamma (γ) chain and the TCR delta (δ) chain in the same sample. In such a case, combinations of forward and reverse primers 10, 20 according to above are used in the same reaction mixture, or the sample is split into two samples and the two clonality determinations are run in parallel.

In fact, it is actually possible to determine clonality of both αβ TCRs and αβ T cells and γδ TCRs and γδ T cells in the same sample according to the present invention.

In a further embodiment, determining lymphocyte clonality comprises determining clonality of BCRs and B cells. In particular embodiments, the M forward primers 10 comprise at least one forward primer 10 per V region of the BCR and the N reverse primers 20 comprise at least one reverse primer 20 per D region of the BCR, or the M forward primers 10 comprise at least one forward primer 10 per D region of the BCR and the N reverse primers 20 comprise at least one reverse primer 20 per J region of the BCR, or the M forward primers 10 comprise at least one forward primer 10 per V region of the BCR and the N reverse primers 20 comprise at least one reverse primer 20 per J region of the BCR.

In fact, it is actually possible to determine clonality of both αβ and/or γδ TCRs and αβ and/or γδ T cells and BCRs and B cells in the same sample according to the present invention.

In a particular embodiment, the M forward primers 10 are multiple different hairpin barcode forward primers 10 comprising at least one hairpin barcode forward primer 10 per variable (V) region of a TCR or BCR. In such a particular embodiment, each target-specific sequence 16 of the hairpin barcode forward primers 10 is preferably complementary to a respective variable (V) region 2 of the TCR or BCR.

In such an embodiment, step S1 of FIG. 1 preferably comprises contacting the sample with the multiple different hairpin barcode forward primers 10 and multiple different reverse primers 20. The multiple different reverse primers 20 comprise at least one reverse primer 20 per joining (J) region 3 of the TCR or BCR. In this embodiment, each reverse primer 20 comprises, from the 5′ end 21 to the 3′ end 27, the adapter sequence 23 and a target-specific sequence 26 complementary to a respective joining (J) region 3 of the TCR or BCR.

In an embodiment, the N reverse primers 20 is a hairpin barcode reverse primer 20 comprising a target-specific sequence 26 complementary to a joining (J) region 3 of the BCR, preferably of an immunoglobulin heavy chain (IGH) of the BCR. In this embodiment, the M forward primers 10 comprise at least one forward primer 10 per variable (V) region 2 of BCR, preferably of the IGH of the BCR.

The monitoring of T and/or B cell clonality has immense importance in the diagnosis and follow-up of various disease, such as hematological diseases and is a fundamental tool for the understanding of antigen specificity in immunity to infections, cancer, and in autoimmunity. Clinically, there is a need for improved methodological specificity and sensitivity to determine the immune repertoire, for example, to detect acute lymphoblastic leukemia and to monitor minimal residual disease. Moreover, in studies of immune disorders such as autoimmunity and tumor immunity, improved immune repertoire analysis will facilitate the identification of involved immune cells, be used to evaluate the efficiency of immunomodulating drugs, and explore their mode of action.

Thus, the lymphocyte clonality as determined according to the present invention is a valuable source of information in diagnosis and monitoring of various diseases, and can thereby be used in disease characterization.

Another aspect of the invention therefore relates to a method of disease characterization. The method comprises determining lymphocyte clonality as disclosed herein of a sample comprising nucleic acid molecules 1 of lymphocytes obtained from a subject. The method also comprises characterizing a disease of the subject based on the determined lymphocyte clonality. In an embodiment, the disease is selected from the group consisting of a hematologic disease, an infectious disease, a cancer disease and an autoimmune disease.

Characterizing a disease as used herein could, in an embodiment, include predicting or determining a likelihood that the subject is suffering from the disease. Hence, information of the lymphocyte clonality, as determined from a sample taken from the subject, can be used to assess whether the subject is likely to suffer from the disease.

For instance, information of lymphocyte clonality could be useful in monitoring infections, such as bacterial infections or virus infections. Hence, in an embodiment, information of lymphocyte clonality as determined according to the invention could be used in monitoring, including diagnosing, a virus infection, such as a coronavirus infection, including, but not limited to, a severe acute respiratory syndrome (SARS) coronavirus 1 (SARS-CoV-1), SARS-CoV-2, Middle East Respiratory Syndrome (MERS) coronavirus (MERS-CoV).

Alternatively, or additionally, characterizing a disease could comprise monitoring disease progression, such as monitoring whether the subject recovers from a disease, whether the subject's condition deteriorates or whether subject's disease condition remains unchanged. Thus, in information of lymphocyte clonality could be used to monitor disease progression and recovery from the disease.

Characterizing a disease could further include evaluating the efficiency of various treatments of a disease, such as administration of drugs, e.g., immunomodulating drugs, and/or determining their mode of action. Hence, information of lymphocyte clonality could then be used to assess how well a subject responds to the treatment and whether the treatment seem to have any effect on the disease or, whether another potentially more effect treatment should be selected instead.

### EXAMPLES

### Example 1

In this example, an ultrasensitive immune repertoire sequencing approach was developed, sequencing the rearranged T-cell receptor delta (TRD) gene in γδ T cells. To minimize the formation of non-specific PCR products, the UMI was protected inside a hairpin loop that opens and closes its secondary structure in a temperature-dependent manner. Primers specific for all V and J genes in the TRD locus were developed and optimized and all possible TRD recombinations were tested and validated individually and as a multiplex assay using synthetic analogs of rearranged immune receptors as well as human DNA. Dynamic range, sensitivity, reproducibility, and specificity were evaluated and the method was applied on both enriched γδ T cells and non-enriched peripheral blood mononuclear cells (PBMC) from healthy donors. The sequencing approach to determine the immune repertoire using targeted PCR based UMIs is sensitive, specific, and reproducible, providing new means to study the immune repertoire of γδ T cells.

### Material and Methods

### Isolation of γδT Cells

Fully anonymized blood samples were obtained from healthy blood donors at the Department of Transfusion Medicine, Sahlgrenska University Hospital. The study was approved by the Regional Research Ethics Committee in Gothenburg (reference numbers: 355-12 and T487-14). Two types of human samples were used, 5 to 10 ml of residual leukocytes (buffy coat) processed using Reveos automated blood processing system (TerumoBCT) from whole blood donated by healthy individuals, and 15 ml of peripheral blood samples in two 9 ml LH Lithium Heparin Vacuette tubes (Greiner Bio-One). The buffy coats were diluted 1:4, and the blood samples 1:1, with sterile phosphate-buffered saline (PBS, pH 7.2) preheated to 22° C. in water bath and then applied on top of Ficoll-Paque PLUS density gradient media (density: 1.077 g/ml, GE Healthcare) preheated to 22° C. in a 50 ml Falcon conical centrifuge tubes (Thermo Fisher Scientific). Next, PBMCs were isolated by density gradient centrifugation (400×g for 20 min at 22° C., low acceleration, and no break) in a Heraeus Megafuge 40R centrifuge (Thermo Fisher Scientific). The PBMC layer was collected in 10 ml conical base tubes (Sarstedt), with remaining volume filled with PBS and then centrifugated (300×g for 5 min at 4° C.). The supernatant was removed, and the cell pellet was resuspended in 1 to 5 ml PBS. The number of viable cells was quantified using Trypan Blue solution (0.4%, Thermo Fisher Scientific) and dye exclusion test, according to the manufacturer's instructions.

γδ T cells were isolated from buffy coat by negative selection using the EasySep Human Gamma/Delta T-cell Isolation Kit (Stemcell Technologies), according to the manufacturer's instructions. The purity of the γδ T-cell enriched buffy coat samples was determined by flow cytometry analysis, comparing the enriched sample with the original isolated PBMCs. In the peripheral blood, the γδ T-cell frequency varied between 1.2% and 12.3% of the total T-cell population, while the frequency of γδ T cells in the enriched preparation was between 85.5% and 90.6%.

### Fluorescence-Activated Cell Sorting (FACS) and Flow Cytometty Analysis

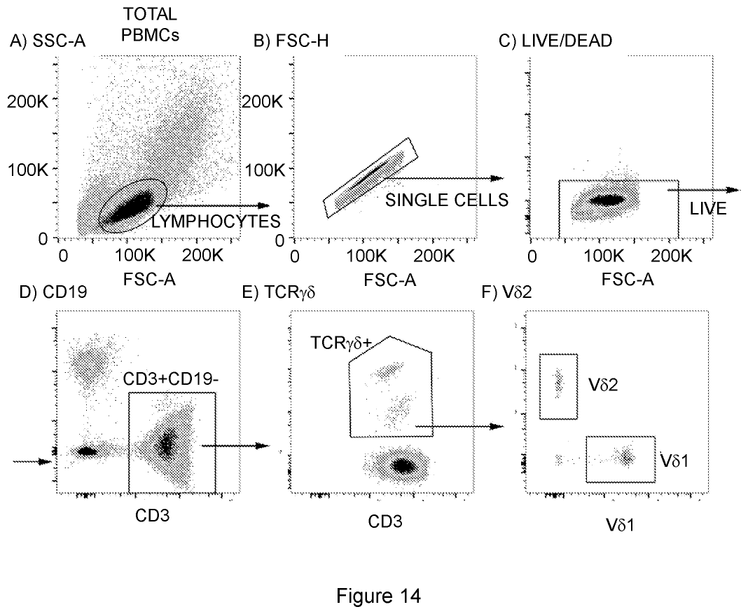

To prepare cells for FACS and flow cytometry analysis, PBMCs were transferred to a 96-well round bottom plate (Thermo Fisher Scientific). The plate was then centrifuged and supernatant was removed. To assess cell viability, the cell pellet was resuspended with 100 μl LIVE/DEAD aqua stain (Life Technologies) diluted 1:1000 in PBS, and then incubated for 15 min (room temperature, protected from light). The cells were centrifuged and washed with 100 μl FACS buffer consisting of PBS supplied with 2% fetal bovine serum (Thermo Fisher Scientific) and 2 mM EDTA (Sigma-Aldrich). To block unspecific antibody bindings, cell pellets were resuspended in 100 μl Fc receptor binding inhibitor (eBioscience) diluted 1:4 in FACS buffer and incubated (15 min at 4° C., protected from light). The supernatant was removed after centrifugation and the cell pellets were resuspended with a 100 μl mix of fluorochrome-conjugated monoclonal antibodies in FACS buffer (25 min at 4° C., in the dark). The following fluorochrome-conjugated monoclonal antibodies were used: anti-CD3 BV786 (clone OKT3, 1:100), anti-CD19 APC-Cy7 (clone HIB19, 1:100), anti-TCRγδ PE (clone B1, 1:50), anti-TCR Vδ2 BV711 (clone B6, 1:50) (all Biolegend) and anti-TCR Vδ1 FITC (clone TS8.2, 1:100) (Thermo Fisher Scientific). After staining, 100 μl of FACS buffer was added. The cells were then centrifuged, and supernatant was removed. The stained samples were washed once with 200 μl FACS buffer and resuspended in 200 μl FACS buffer before flow cytometry analysis. For the staining process, all centrifugation steps were performed at 300×g, for 3 min at 4° C. in a refrigerated Heraeus Megafuge 40R centrifuge. The gating strategy is shown in FIG. 14. The γδ T cells in peripheral blood samples were sorted into 20 μl FACS buffer using the BD FACSAria Fusion cell sorter (BD Biosciences). Purity of sorted γδ T cells was between 88.8 and 93.4% of total events and between 95.2% and 97.3% of total T-cell counts. The buffy coat samples were analyzed using the BD LSRFortessa X-20 cell analyzer (BD Biosciences). The flow cytometric data were analyzed using the FlowJo 10.5.3 software (TreeStar).

### DNA Extraction

DNA from human samples and T47D breast cancer cell lines (ATCC) was extracted using the QIAamp DNA Blood Mini Kit (Qiagen), according to the manufacturer's instructions and quantified using dsDNA HS Assay Kit with a Qubit 3 (both Thermo Fischer Scientific). DNA extracted from peripheral blood samples were concentrated using DNA Clean & Concentrator-5 kit (Zymo Research).

### Synthetic Molecules

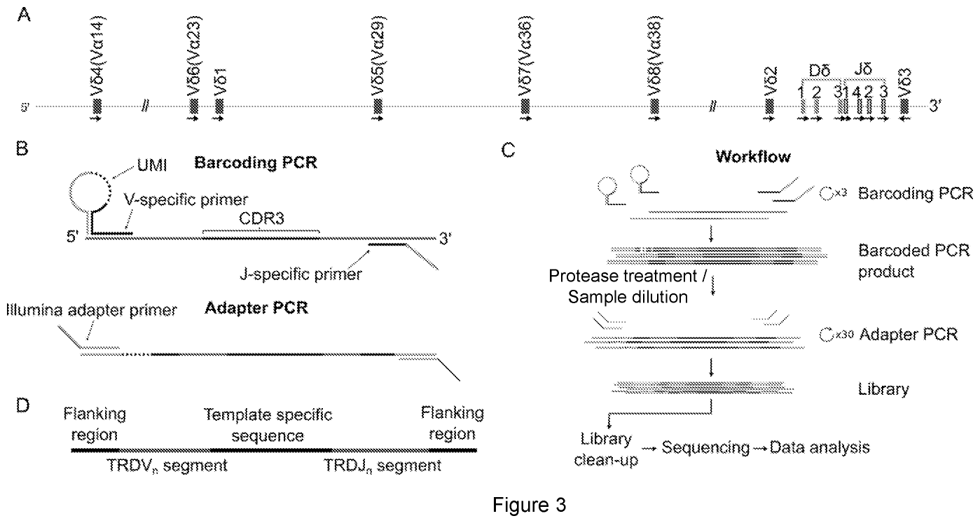

32 specific gBlock molecules (IDT, Table 1) were designed for each combination of the TRDV and TRDJ genes. The sequence design is schematically shown in FIG. 3D. gBlocks were reconstituted in TE-buffer (pH 8.0, Thermo Fisher Scientific), and their concentrations were quantified using the dsDNA HS Assay Kit with a Qubit 3. All further dilution of synthetic molecules was made using a buffer containing 1 μg/μl bovine serum albumin supplied in 2.5% glycerol (Thermo Fischer Scientific).

TABLE 1
Sequences of synthetic reference molecules

| | | SEQ |
|---|---|---|
| | | ID |
| Primer | Primer sequence (5′→3′) | NO: |
| TRDV4/ | 5′- | 28 |
| TRAV14- | tcggtggttctctaactactatcACCAGCAAAATGCAACAGAAGGT | |
| TRDJ1 | CGCTACTCATTGAATTT [symbol] CACCTTG | |
| | TCATCTCCGCTTCACAACTGGGGGACTCAGCAATGTACTTCTGTGC | |
| | AATGAGAGAGGGagctagctagGTTGTGTACTagctagctagACAC | |
| | CGATAAACTCATCTTTGGAA [symbol] GAACCA | |
| | Accaacgaacatgtaactctca-3′ | |
| TRDV4/ | 5′- | 29 |
| TRAV14- | tcggtggttctctaactactatcACCAGCAAAATGCAACAGAAGGT | |
| TRDJ2 | CGCTACTCATTGAATTT [symbol] CAACCTTG | |
| | TCATCTCCGCTTCACAACTGGGGGACTCAGCAATGTACTTCTGTGC | |
| | AATGAGAGAGGGagctagctagAAATCGGTCTagctagctagCTTT | |
| | GACAGCACAACTCTTCTTTGGAAAG [symbol] A | |
| | CCAGccaacgaacatgtaactctca-3′ | |
| TRDV4/ | 5′- | 30 |
| TRAV14- | tcggtggttctctaactactatcACCAGCAAAATGCAACAGAAGGT | |
| TRDJ3 | CGCTACTCATTGAATTT [symbol] CAAGCTTG | |
| | TCATCTCCGCTTCACAACTGGGGGACTCAGCAATGTACTTCTGTGC | |
| | AATGAGAGAGGGagctagctagTATGCCTGAAagctagctagCTCC | |
| | TGGGACACCCGACAGATGTTTT [symbol] CG | |
| | TGGAGCCCCccaacgaacatgtaactctca-3′ | |
| TRDV4/ | 5′- | 31 |
| TRAV14- | tcggtggttctctaactactatcACCAGCAAAATGCAACAGAAGGT | |
| TRDJ4 | CGCTACTCATTGAATTT [symbol] CAAGCTTG | |
| | TCATCTCCGCTTCACAACTGGGGGACTCAGCAATGTACTTCTGTGC | |
| | AATGAGAGAGGGagctagctagATGGGTCATTagctagctagCCAG | |
| | ACCCCTGATCTTTGGCA [symbol] ACAACcc | |
| | aacgaacatgtaactctca-3′ | |
| TRDV6/ | 5′- | 32 |
| TRAV23- | tcggtggttctctaactactatcTTATTGATAGCCATACGTCCAGA | |
| TRDJ1 | TGTGAGTGAAAAGAAAGAAGGAAGATTCACAATCTCCTTCAATAAA | |
| | AGTGCCAA [symbol] ATTCCCAGCCTGGAG | |
| | ACTCAGCCACCTACTTCTGTGCAGCAAGCAagctagctagCCCCTT | |
| | TAGTagctagctagACACCGATAAACTCATCTTTGGAA [symbol] | |
| | [symbol] GAACCAAccaacgaacatgtaactctca-3′ | |
| TRDV6/ | 5′- | 33 |
| TRAV23- | tcggtggttctctaactactatcTTATTGATAGCCATACGTCCAGA | |
| TRDJ2 | TGTGAGTGAAAAGAAAGAAGGAAGATTCACAATCTCCTTCAATAAA | |
| | AGTGCCAA [symbol] ATTCCCAGCCTGGAG | |
| | ACTCAGCCACCTACTTCTGTGCAGCAAGCAagctagctagGAAATC | |
| | GTAGagctagctagCTTTGACAGCACAACTCTTCTTTGGAAAG [symbol] | |
| | [symbol] ACCAGccaacgaacatgtaactctca-3′ | |
| TRDV6/ | 5′- | 34 |
| TRAV23- | tcggtggttctctaactactatcTTATTGATAGCCATACGTCCAGA | |
| TRDJ3 | TGTGAGTGAAAAGAAAGAAGGAAGATTCACAATCTCCTTCAATAAA | |
| | AGTGCCAA [symbol] ATTCCCAGCCTGGAG | |
| | ACTCAGCCACCTACTTCTGTGCAGCAAGCAagctagctagTCATTT | |
| | TGCGagctagctagCTCCTGGGACACCCGACAGATGTTTT [symbol] | |
| | [symbol] CGTGGAGCCCCccaacgaacatgtaactct | |
| | ca-3′ | |
| TRDV6/ | 5′- | 35 |
| TRAV23- | tcggtggttctctaactactatcTTATTGATAGCCATACGTCCAGA | |
| TRDJ4 | TGTGAGTGAAAAGAAAGAAGGAAGATTCACAATCTCCTTCAATAAA | |
| | AGTGCCAA [symbol] ATTCCCAGCCTGGAG | |
| | ACTCAGCCACCTACTTCTGTGCAGCAAGCAagctagctagGGTGTG | |
| | CAAAagctagctagCCAGACCCCTGATCTTTGGCA [symbol] | |
| | [symbol] ACAACccaacgaacatgtaactctca-3′ | |
| TRDV5/ | 5′- | 36 |
| TRAV29- | tcggtggttctctaactactatcATAAAAATGAAGATGGAAGATTC | |
| TRDJ1 | ACTGTTTT [symbol] TCTCTCTGCACATTG | |
| | TGCCCTCCCAGCCTGGAGACTCTGCAGTGTACTTCTGTGCAGCAAG | |
| | CGagctagctagAATTCTATTTagctagctagACACCGATAAACTC | |
| | ATCTTTGGAA [symbol] GAACCAAccaacgaac | |
| | atgtaactctca-3′ | |
| TRDV5/ | 5′- | 37 |
| TRAV29- | tcggtggttctctaactactatcATAAAAATGAAGATGGAAGATTC | |
| TRDJ2 | ACTGTTTT [symbol] TCTCTCTGCACATTG | |
| | TGCCCTCCCAGCCTGGAGACTCTGCAGTGTACTTCTGTGCAGCAAG | |
| | CGagctagctagTATTTTGCTTagctagctagCTTTGACAGCACAA | |
| | CTCTTCTTTGGAAAG [symbol] ACCAGccaacg | |
| | aacatgtaactctca-3′ | |
| TRDV5/ | 5′- | 38 |
| TRAV29- | tcggtggttctctaactactatcATAAAAATGAAGATGGAAGATTC | |
| TRDJ3 | ACTGTTTT [symbol] TCTCTCTGCACATTG | |
| | TGCCCTCCCAGCCTGGAGACTCTGCAGTGTACTTCTGTGCAGCAAG | |
| | CGagctagctagCCGGCCGGGGagctagctagCTCCTGGGACACCC | |
| | GACAGATGTTTT [symbol] CGTGGAGCCCCc | |
| | caacgaacatgtaactctca-3′ | |
| TRDV5/ | 5′- | 39 |
| TRAV29- | tcggtggttctctaactactatcATAAAAATGAAGATGGAAGATTC | |
| TRDJ4 | ACTGTTTT [symbol] TCTCTCTGCACATTG | |
| | TGCCCTCCCAGCCTGGAGACTCTGCAGTGTACTTCTGTGCAGCAAG | |
| | CGagctagctagGGTTACCCTCagctagctagCCAGACCCCTGATC | |
| | TTTGGCA [symbol] ACAACccaacgaacatg | |
| | taactctca-3′ | |
| TRDV7/ | 5′- | 40 |
| TRAV36- | tcggtggttctctaactactatcGTCAGGAAGACTAAGTAGCATAT | |
| TRDJ1 | TAGATAAGAAAGAACTTTTCAGCATCCTGAACATCACAGCCACCC [symbol] | |
| | [symbol] CTACCTCTGTGCTGTGGAGGagctagct | |
| | agTTTAAGTTAAagctagctagACACCGATAAACTCATCTTTGGAA | |
| | [symbol] GAACCAAccaacgaacatgtaactct | |
| | ca-3′ | |
| TRDV7/ | 5′- | 41 |
| TRAV36- | tcggtggttctctaactactatcGTCAGGAAGACTAAGTAGCATAT | |
| TRDJ2 | TAGATAAGAAAGAACTTTTCAGCATCCTGAACATCACAGCCACCC [symbol] | |
| | [symbol] CTACCTCTGTGCTGTGGAGGagctagct | |
| | agTGTACTCAGGCagctagctagCTTTGACAGCACAACTCTTCTTT | |
| | GGAAAG [symbol] ACCAGccaacgaacatgtaa | |
| | ctctca-3′ | |
| TRDV7/ | 5′- | 42 |
| TRAV36- | tcggtggttctctaactactatcGTCAGGAAGACTAAGTAGCATAT | |
| TRDJ3 | TAGATAAGAAAGAACTTTTCAGCATCCTGAACATCACAGCCACCC [symbol] | |
| | [symbol] CTACCTCTGTGCTGTGGAGGagctagct | |
| | agTCATGCTGGGagctagctagCTCCTGGGACACCCGACAGATGTT | |
| | TT [symbol] CGTGGAGCCCCccaacgaacat | |
| | gtaactctca-3′ | |
| TRDV7/ | 5′- | 43 |
| TRDJ4 | tcggtggttctctaactactatcGTCAGGAAGACTAAGTAGCATAT | |
| TRAV36- | TAGATAAGAAAGAACTTTTCAGCATCCTGAACATCACAGCCACCC [symbol] | |
| | [symbol] CTACCTCTGTGCTGTGGAGGagctagct | |
| | agCTCGGTGCAAagctagctagCCAGACCCCTGATCTTTGGCA [symbol] | |
| | [symbol] ACAACccaacgaacatgtaactctca- | |
| | 3′ | |
| TRDV8/ | 5′- | 44 |
| TRAV38-2- | tcggtggttctctaactactatcAGAATCGTTTCTCTGTGAACTTC | |
| TRDJ1 | CAGAAAGCAGCCAAATCCTTCAGTCTCAAGATCTCAGACTCACAGC | |
| | TG [symbol] TTCTGTGCTTATAGGAGCGagctagc | |
| | tagGAAAGGGTTCagctagctagACACCGATAAACTCATCTTTGGA | |
| | A [symbol] GAACCAAccaacgaacatgtaactc | |
| | tca-3′ | |
| TRDV8/ | 5′- | 45 |
| TRAV38-2- | tcggtggttctctaactactatcAGAATCGTTTCTCTGTGAACTTC | |
| TRDJ2 | CAGAAAGCAGCCAAATCCTTCAGTCTCAAGATCTCAGACTCACAGC | |
| | TG [symbol] TTCTGTGCTTATAGGAGCGagctagc | |
| | tagTTTTTACCCAagctagctagCTTTGACAGCACAACTCTTCTTT | |
| | GGAAAG [symbol] ACCAGccaacgaacatgtaa | |
| | ctctca-3′ | |
| TRDV8/ | 5′- | 46 |
| TRAV38-2- | tcggtggttctctaactactatcAGAATCGTTTCTCTGTGAACTTC | |
| TRDJ3 | CAGAAAGCAGCCAAATCCTTCAGTCTCAAGATCTCAGACTCACAGC | |
| | TG [symbol] TTCTGTGCTTATAGGAGCGagctagc | |
| | tagGCCCTTTTGGagctagctagCTCCTGGGACACCCGACAGATGT | |
| | TTT [symbol] CGTGGAGCCCCccaacgaaca | |
| | tgtaactctca-3′ | |
| TRDV8/ | 5′- | 47 |
| TRAV38-2- | tcggtggttctctaactactatcAGAATCGTTTCTCTGTGAACTTC | |
| TRDJ4 | CAGAAAGCAGCCAAATCCTTCAGTCTCAAGATCTCAGACTCACAGC | |
| | TG [symbol] TTCTGTGCTTATAGGAGCGagctagc | |
| | tagATTTTTTCCCagctagctagCCAGACCCCTGATCTTTGGCA [symbol] | |
| | [symbol] ACAACccaacgaacatgtaactctca- | |
| | 3′ | |
| TRDV1- | 5′- | 71 |
| TRDJ1 | tcggtggttctctaactactatcCTGTCAACTTCAAGAAAGCA [symbol] | |
| | [symbol] CATTTCAGCCTTACAGCTAGAAGATTCAG | |
| | CAAAGTACTTTTGTGCTCTTGGGGAACTagctagctagAAGATAGA | |
| | ATagctagctagACACCGATAAACTCATCTTTGGAA [symbol] | |
| | [symbol] GAACCAAccaacgaacatgtaactctca-3′ | |
| TRDV1- | 5′- | 72 |
| TRDJ2 | tcggtggttctctaactactatcCTGTCAACTTCAAGAAAGCA [symbol] | |
| | [symbol] CATTTTCAGCCTTACAGCTAGAAGATTCAG | |
| | CAAAGTACTTTTGTGCTCTTGGGGAACTagctagctagGTCAAACT | |
| | GagctagctagCTTTGACAGCACAACTCTTCTTTGGAAAG [symbol] | |
| | [symbol] ACCAGccaacgaacatgtaactctca-3′ | |
| TRDV1- | 5′- | 73 |
| TRDJ3 | tcggtggttctctaactactatcCTGTCAACTTCAAGAAAGCA [symbol] | |
| | [symbol] CATTTCAGCCTTACAGCTAGAAGATTCAG | |
| | CAAAGTACTTTTGTGCTCTTGGGGAACTagctagctagTTATCGAT | |
| | GagctagctagCTCCTGGGACACCCGACAGATGTTTT [symbol] | |
| | [symbol] CGTGGAGCCCCccaacgaacatgtaactctca- | |
| | 3′ | |
| TRDV1- | 5′- | 74 |
| TRDJ4 | tcggtggttctctaactactatcCTGTCAACTTCAAGAAAGCA [symbol] | |
| | [symbol] CATTTCAGCCTTACAGCTAGAAGATTCAG | |
| | CAAAGTACTTTTGTGCTCTTGGGGAACTagctagctagATCGTAGT | |
| | GagctagctagCCAGACCCCTGATCTTTGGCA [symbol] | |
| | [symbol] ACAACccaacgaacatgtaactctca-3′ | |
| TRDV2- | 5′- | 75 |
| TRDJ1 | tcggtggttctctaactactatcTTCCAAGGTGACATTGATAT [symbol] | |
| | [symbol] AGATACTTGCACCATCAGAGAGAGAT | |
| | GAAGGGTCTTACTACTGTGCCTGTGACACCagctagctagGGATCG | |
| | ACTagctagctagACACCGATAAACTCATCTTTGGAA [symbol] | |
| | [symbol] GAACCAAccaacgaacatgtaactctca-3′ | |
| TRDV2- | 5′- | 76 |
| TRDJ2 | tcggtggttctctaactactatcTTCCAAGGTGACATTGATAT [symbol] | |
| | [symbol] AGATACTTGCACCATCAGAGAGAGAT | |
| | GAAGGGTCTTACTACTGTGCCTGTGACACCagctagctagCATGAT | |
| | TCAagctagctagCTTTGACAGCACAACTCTTCTTTGGAAAG [symbol] | |
| | [symbol] ACCAGccaacgaacatgtaactctca-3′ | |
| TRDV2- | 5′- | 77 |
| TRDJ3 | tcggtggttctctaactactatcTTCCAAGGTGACATTGATAT [symbol] | |
| | [symbol] AGATACTTGCACCATCAGAGAGAGAT | |
| | GAAGGGTCTTACTACTGTGCCTGTGACACCagctagctagAAATCG | |
| | TAAagctagctagCTCCTGGGACACCCGACAGATGTTTT [symbol] | |
| | [symbol] CGTGGAGCCCCccaacgaacatgtaactctc | |
| | a-3′ | |
| TRDV2- | 5′- | 78 |
| TRDJ4 | tcggtggttctctaactactatcTTCCAAGGTGACATTGATAT [symbol] | |
| | [symbol] AGACACTTGCACCATCAGAGAGAGAT | |
| | GAAGGGTCTTACTACTGTGCCTGTGACACCagctagctagTTAGGT | |
| | ACTagctagctagCCAGACCCCTGATCTTTGGCA [symbol] | |
| | [symbol] ACAACccaacgaacatgtaactctca-3′ | |
| TRDV3- | 5′- | 79 |
| TRDJ1 | tcggtggttctctaactactatcTGTAAACAAATGAAACTACT [symbol] | |
| | [symbol] CACAGTAAGAATCATCTTTTCTTC | |
| | ATATCAGGGCAGAGGATATACAACAAAACAGGGTTCCTAAGATCTC | |
| | AGAGACTATCTGACGGTTCTAATGAAAGAagctagctagACCTGAT | |
| | GTagctagctagACACCGATAAACTCATCTTTGGAA [symbol] | |
| | [symbol] GAACCAAccaacgaacatgtaactctca-3′ | |
| TRDV3- | 5′- | 80 |
| TRDJ2 | tcggtggttctctaactactatcTGTAAACAAATGAAACTACT [symbol] | |
| | [symbol] CACAGTAAGAATCATCTTTTCTTC | |
| | ATATCAGGGCAGAGGATATACAACAAAACAGGGTTCCTAAGATCTC | |
| | AGAGACTATCTGACGGTTCTAATGAAAGAagctagctagTAGCATG | |
| | CAagctagctagCTTTGACAGCACAACTCTTCTTTGGAAAG [symbol] | |
| | [symbol] ACCAGccaacgaacatgtaactctca-3′ | |
| TRDV3- | 5′- | 81 |
| TRDJ3 | tcggtggttctctaactactatcTGTAAACAAATGAAACTACT [symbol] | |
| | [symbol] CACAGTAAGAATCATCTTTTCTTC | |
| | ATATCAGGGCAGAGGATATACAACAAAACAGGGTTCCTAAGATCTC | |
| | AGAGACTATCTGACGGTTCTAATGAAAGAagctagctagCAGTCAC | |
| | TAagctagctagCTCCTGGGACACCCGACAGATGTTTT [symbol] | |
| | [symbol] CGTGGAGCCCCccaacgaacatgtaactctca- | |
| | 3′ | |
| TRDV3- | 5′- | 82 |
| TRDJ4 | tcggtggttctctaactactatcTGTAAACAAATGAAACTACT [symbol] | |
| | [symbol] CACAGTAAGAATCATCTTTTCTTC | |
| | ATATCAGGGCAGAGGATATACAACAAAACAGGGTTCCTAAGATCTC | |
| | AGAGACTATCTGACGGTTCTAATGAAAGAagctagctagATCGATG | |
| | CTagctagctagCCAGACCCCTGATCTTTGGCA [symbol] | |
| | [symbol] ACAACccaacgaacatgtaactctca-3′ | |

Double underline-TRDV segment; Single underline-TRDJ segment; Bold-primer binding site

### Primer Design

To capture the full diversity of the T-cell receptor 6 repertoire, target-specific primers were designed for all eight variable (TRDV) and four joining (TRDJ) genes associated to the TRD locus in the IMGT/GENE database (Giudicelli et al., 2005) (FIG. 3A). Primers were designed using NCBI Primer-Blast (Ye et al., 2012) as previously described (St\dot{a}hlberg et al., 2017). Forward primers targeted the downstream part of the TRDV genes, and the reverse primer targeted the downstream part of the TRDJ genes (FIG. 3B). Primers were designed to bind the non-rearranged parts of each segment amplifying the CDR3 region (Table 2).

TABLE 2
Primer sequence information

| | | | | | |
|---|---|---|---|---|---|
| | | SEQ | | | |
| | | ID | | | |
| Primer | Target sequence (5′-3′) | NO: | Strand¹ | Start² | Stop² |

TargetV-region forward primer

| | | | | | |
|---|---|---|---|---|---|
| TRDV1 | GCGAAATCCGTCGCCTTAAC | 1 | F | 22096543 | 22096562 |
| TRDV2 | ACTTGCACCATCAGAGAGAGATG | 2 | F | 22422991 | 22423013 |
| TRDV3 | TCCAGTAAGGACTGAAGACAGTG | 3 | R | 22469085 | 22469063 |
| TRDV4/TRAV14 | CCAGAAGGCAAGAAAATCCGC | 4 | F | 21924565 | 21924585 |
| TRDV5/TRAV29 | CTTAAACAAAAGTGCCAAGCACC | 5 | F | 22163785 | 22163807 |
| TRDV6/TRAV23 | GCAGTTCTCATCGCATATCATGG | 6 | F | 22086894 | 2208691 |
| TRDV7/TRAV36 | AGACCGGAGACTCGGCCAT | 7 | F | 22227216 | 22227234 |
| TRDV8/TRAV38- | GGGGATGCCGCGATGTAT | 8 | F | 22281712 | 22281729 |
| 2 | | | | | |

Target J-region reverse primer

| | | | | | |
|---|---|---|---|---|---|
| TRDJ1 | CACAGTCACACGGGTTCCTT | 9 | R | 22450132 | 22450113 |
| TRDJ2 | CGATGAGTTGTGTTCCCTTTCCAA | 10 | R | 22456733 | 22456710 |
| TRDJ3 | AGTTTGATGCCAGTTCCGAAA | 11 | R | 22459142 | 22459122 |
| TRDJ4 | GTTGTACCTCCAGATAGGTTCCT | 12 | R | 22455293 | 22455271 |

Hairpin barcode forward primer
5′-GGACACTCTTTCCCTACACGACGCTCTTCCGATCTNNNNNNNNNNNNATGGGAAAGAGTGTC
C-V-region forward primer-3′ (SEQ ID NO: 13)
Reverse primer
5′-GTGACTGGAGTTCAGACGTGTGCTCTTCCGATCT-J-region reverse primer-3′
(SEQ ID NO: 14)
¹F: Forward; R: Reverse
²Nucleotide position from GRCh38/hg38, chromosome 14 (Kent et al., 2002)

TABLE 3
ILLUMINA ® index primers

| | | SEQ ID |
|---|---|---|
| Primer | Primer sequence (5′-3′) | NO: |
| Universal | AATGATACGGCGACCACCGAGATCTAGACTCTTTCCCTACACGA | 17 |
| forward adapter | CGCTCTTCCGATCT | |
| primer | | |
| Reverse index 1 | CAAGCAGAAGACGGCATACGAGATCGTGATGTGACTGGAGTTCA | 18 |
| adapter primer | GACGTGTGCTCTTCCGATCT | |
| Reverse index 2 | CAAGCAGAAGACGGCATACGAGATACATCGGTGACTGGAGTTCA | 19 |
| adapter primer | GACGTGTGCTCTTCCGATCT | |
| Reverse index 3 | CAAGCAGAAGACGGCATACGAGATGCCTAAGTGACTGGAGTTCA | 20 |
| adapter primer | GACGTGTGCTCTTCCGATCT | |
| Reverse index 4 | CAAGCAGAAGACGGCATACGAGATTGGTCAGTGACTGGAGTTCA | 21 |
| adapter primer | GACGTGTGCTCTTCCGATCT | |
| Reverse index 5 | CAAGCAGAAGACGGCATACGAGATCACTGTGTGACTGGAGTTCA | 22 |
| adapter primer | GACGTGTGCTCTTCCGATCT | |
| Reverse index 6 | CAAGCAGAAGACGGCATACGAGATATTGGCGTGACTGGAGTTCA | 23 |
| adapter primer | GACGTGTGCTCTTCCGATCT | |
| Reverse index 7 | CAAGCAGAAGACGGCATACGAGATGATCTGGTGAGTGGAGTTCA | 24 |
| adapter primer | GACGTGTGCTCTTCCGATCT | |
| Reverse index 8 | CAAGCAGAAGACGGCATACGAGATTCAAGTGTGACTGGAGTTCA | 25 |
| adapter primer | GACGTGTGCTCTTCCGATCT | |
| Reverse index 9 | CAAGCAGAAGACGGCATACGAGATCTGATCGTGACTGGAGTTCA | 26 |
| adapter primer | GACGTGTGCTCTTCCGATCT | |
| Reverse index | CAAGCAGAAGACGGCATACGAGATAAGCTAGTGACTGGAGTTCA | 27 |
| 10 adapter | GACGTGTGCTCTTCCGATCT | |
| primer | | |

Sample index barcode index are shown in bold.

### Assay Validation

Each primer pair combination of TRDV and TRDJ was tested using a standard curve, ranging from 40,000 to 12.8 synthetic gBlock molecules in steps of five. Quantitative PCR (qPCR) was performed in a CFX384 Touch Real-Time PCR Detection System (BioRad) in 10 μl reactions, containing 1×TATAA SYBR GrandMaster Mix (TATAA Biocenter), 400 nM of each primer (desalted, Sigma-Aldrich, Table 2) and 2 μl diluted gBlocks. Final reaction concentrations are shown. The following temperature profile was used: 98° C. for 3 min, followed by 50 cycles of amplification (98° C. for 10 sec, 60° C. for 30 sec, 72° C. for 20 sec) and a melting curve ranging from 60° C. to 95° C., 0.2° C./sec increments. No template controls and genomic DNA from the breast cancer cell line T47D was included for each assay. The T47D genomic control was used to check for amplification of non-rearranged products. Cycle of quantification values were determined using regression using the CFX Manager Software version 3.1 (BioRad). PCR product was assessed on a Fragment Analyzer using the dsDNA 35-5000 bp Reagent Kit (Both Advanced Analytical). Validated target primers were then used to design primers that incorporate both UMI and adapter sequences (Table 2).

### Barcoding PCR and Library Construction

DNA were barcoded in a 10 μl reaction, containing 0.2 U Phusion HF polymerase, 1× Phusion High-Fidelity Buffer (both Thermo Fisher Scientific), 0.2 mM dNTP (Sigma-Aldrich), 0.5 M L-carnitine inner salt (Sigma-Aldrich), 40 nM of each barcode primer (PAGE purified, IDT) (Table 2) and target DNA. The following temperature program was used on a T100 Thermal cycler (BioRad): 98° C. for 30 sec, 3 cycles of amplification (98° C. for 10 sec, 62° C. for 6 min, 72° C. for 30 sec, all ramping rate of 4° C./sec), and 65° C. for 15 min and 95° C. for 15 min. Twenty microliters of 45 ng/μl Streptomyces griseus protease (Sigma-Aldrich) dissolved in TE-buffer (pH 8.0) was added to each well at the start of the 15 min incubation step at 65° C. to degrade the polymerase, reducing the formation of non-specific PCR products. ILLUMINA© adapters were added in a second PCR step (Table 3). Ten microliters of barcoded PCR product was amplified in a 40 μl reaction containing, 1×Q5 Hot Start High-Fidelity Master Mix (New England BioLabs) and 400 nM of each ILLUMINA© Adapter index primer (desalted, Sigma-Aldrich, Table 3) using the following thermal program on a T100 Thermal cycler; 98° C. for 3 min, to 40 cycles of amplification (98° C. for 10 sec, 80° C. for 1 sec, 72° C. for 30 sec, 76° C. for 30 sec, all ramping rate of 0.2° C./sec). Final reaction concentrations are shown for each PCR.

To quantify the amount of barcoded DNA we used qPCR on a CFX384 Touch Real-Time PCR Detection System. Two microliters PCR product from the barcoding reaction was used in a 10 μl reaction, containing a final concentration of 1×TATAA SYBR GrandMaster Mix (TATAA Biocenter) and 400 nM of each ILLUMINA® Index adapter (Table 3). The following temperature program was used: 98° C. for 3 min, 40 cycles of amplification (98° C. for 10 sec, 80° C. for 1 sec, 72° C. for 30 sec, 76° C. for sec, all with ramping at 0.2° C./sec), and melting curve ranging from 70° C. to 90° C., 0.2° C./sec increments.

Libraries were purified using the Agencourt AMPure XP system (Beckman Coulter), according to the manufacturer's instructions with a bead to sample ratio of 1:1. Library quality and quantification were assessed on a Fragment Analyzer using the HS NGS Fragment DNF-474 kit (both Agilent), according to the manufacturer's instructions.

Final quantification of library pool was performed by qPCR using NEBNext Library Quant Kit (New England Biolabs), according to manufacturer's instructions. Sequencing was performed on a Miniseq using midi output reagent KIT (300 cycles) with 20% added Phix control v3 (both ILLUMINA®) and 1 pM library. We performed 150 cycles of paired-end sequencing.

### Data Analysis

Sequencing data were processed using Molecular Identifier Guided Error Correction (MIGEC) pipeline using only paired-end reads and overlap-max-offset set to 100 (Shugay et al., 2014). In summary, UMIs were extracted from raw sequencing reads. Data were then grouped according to their UMI sequences and assembled to consensus reads with applied error correction. In the nomenclature used here, all corrected reads with identical sequences including the UMI form a UMI family that corresponds to one rearrangement and all UMI families with identical TRD sequence add up to a clonotype. For analysis of gBlock molecules, a fixed threshold of at least 2 reads per UMI family was used. For biological samples, the automatic threshold of MIGEC was used. CDR3 sequence was extracted from the consensus read and variable, diversity and joining segments were determined by the software. For human samples, results were filtered to only include productive TRD rearrangements. As the barcoding step amplifies on average two unique barcodes per original molecule, each UMI family count was divided by two to determine the number of clonotypes. One nanogram of human genomic DNA was assumed to contain 278 haploid genomes. Consequently, 139 productive CDR3 TRD molecules per nanogram of DNA was expected, since only one allele is commonly productive. Post-processing was performed using modified scripts from VDJtools (Shugay et al., 2015) and TcR R programming package (Nazarov et al., 2015).

### Results

### Development of a Sequencing Approach Targeting the 8 Chain in γδ T Cells Using Unique Molecular Identifiers

To develop an ultrasensitive sequencing approach of the rearranged T-cell receptor delta (TRD) locus in γδ T cells (FIG. 3A), targeted sequencing using UMIs was applied. Target primers were designed for eight TRDV genes and four TRDJ genes (Table 2). The 12 nucleotides long UMI was incorporated between the target primer and the adapter sequence. The strategy was based on protecting the UMI at low temperature in a hairpin structure, minimizing the formation of non-specific PCR products. The method consists of two rounds of PCR. In the first PCR step, all target DNA was barcoded in three cycles of amplification. In the second PCR, the barcoded DNA was amplified with ILLUMINA© adapter primers (FIGS. 3B, 3C and Table 3).

To validate the efficiency of each target primer combination, 32 synthetic gBlock DNA molecules containing the target gene segment of each primer combination (FIG. 3D and Table 1) were used. All target primer pairs showed >90% PCR efficiency using quantitative PCR (qPCR) (Table 4). The specificity of all target primer pair combinations was also tested on genomic DNA from breast cancer cell line T47D that should have the TRD locus in germline configuration, and none of the primer pair combinations produced specific PCR products (data not shown).

TABLE 4
Efficiency of target primers evaluated using qPCR

| | Target | Efficiency | | | |
|---|---|---|---|---|---|
| Target TRDV | TRDJ | (%) | Slope | Y-Intercept | R² |
| TRDV1 | TRDJ1 | 100.7 | −3.31 | 33.3 | 0.998 |
| TRDV1 | TRDJ2 | 92.8 | −3.51 | 33.8 | 0.965 |
| TRDV1 | TRDJ3 | 102.6 | −3.26 | 33.7 | 0.997 |
| TRDV1 | TRDJ4 | 95.3 | −3.44 | 34.5 | 0.996 |
| TRDV2 | TRDJ1 | 101.7 | −3.28 | 31.4 | 0.999 |
| TRDV2 | TRDJ2 | 101.2 | −3.29 | 34.1 | 0.996 |
| TRDV2 | TRDJ3 | 101.9 | −3.28 | 33.6 | 0.999 |
| TRDV2 | TRDJ4 | 101.6 | −3.29 | 34.2 | 0.998 |
| TRDV3 | TRDJ1 | 101.7 | −3.28 | 33.7 | 0.997 |
| TRDV3 | TRDJ2 | 96.7 | −3.40 | 34.7 | 0.994 |
| TRDV3 | TRDJ3 | 93.6 | −3.48 | 35.6 | 0.998 |
| TRDV3 | TRDJ4 | 99.0 | −3.35 | 34.3 | 1 000 |
| TRDV4/TRAV14 | TRDJ1 | 101.0 | −3.30 | 34.2 | 0.999 |
| TRDV4/TRAV14 | TRDJ2 | 99.3 | −3.34 | 33.7 | 0.998 |
| TRDV4/TRAV14 | TRDJ3 | 100.2 | −3.32 | 34.0 | 0.998 |
| TRDV4/TRAV14 | TRDJ4 | 99.6 | −3.33 | 34.0 | 0.997 |
| TRDV5/TRAV29 | TRDJ1 | 95.6 | −3.43 | 34.7 | 0.998 |
| TRDV5/TRAV29 | TRDJ2 | 96.7 | −3.40 | 34.9 | 0.999 |
| TRDV5/TRAV29 | TRDJ3 | 95.5 | −3.43 | 35.2 | 0.999 |
| TRDV5/TRAV29 | TRDJ4 | 100.0 | −3.32 | 34.5 | 0.999 |
| TRDV6/TRAV23 | TRDJ1 | 94.8 | −3.45 | 34.5 | 0.994 |
| TRDV6/TRAV23 | TRDJ2 | 99.8 | −3.33 | 34.2 | 0.998 |
| TRDV6/TRAV23 | TRDJ3 | 104.6 | −3.22 | 33.9 | 0.998 |
| TRDV6/TRAV23 | TRDJ4 | 98.8 | −3.35 | 34.2 | 0.998 |
| TRDV7/TRAV36 | TRDJ1 | 98.6 | −3.36 | 34.3 | 0.995 |
| TRDV7/TRAV36 | TRDJ2 | 106.5 | −3.18 | 33.7 | 0.997 |
| TRDV7/TRAV36 | TRDJ3 | 91.3 | −3.55 | 36.1 | 0.994 |
| TRDV7/TRAV36 | TRDJ4 | 95.4 | −3.44 | 35.0 | 0.993 |
| TRDV8/TRAV38-2 | TRDJ1 | 105.6 | −3.20 | 33.5 | 0.999 |
| TRDV8/TRAV38-2 | TRDJ2 | 103.4 | −3.24 | 34.2 | 0.998 |
| TRDV8/TRAV38-2 | TRDJ3 | 98.1 | −3.37 | 35.7 | 0.999 |
| TRDV8/TRAV38-2 | TRDJ4 | 101.7 | −3.28 | 34.3 | 0.998 |

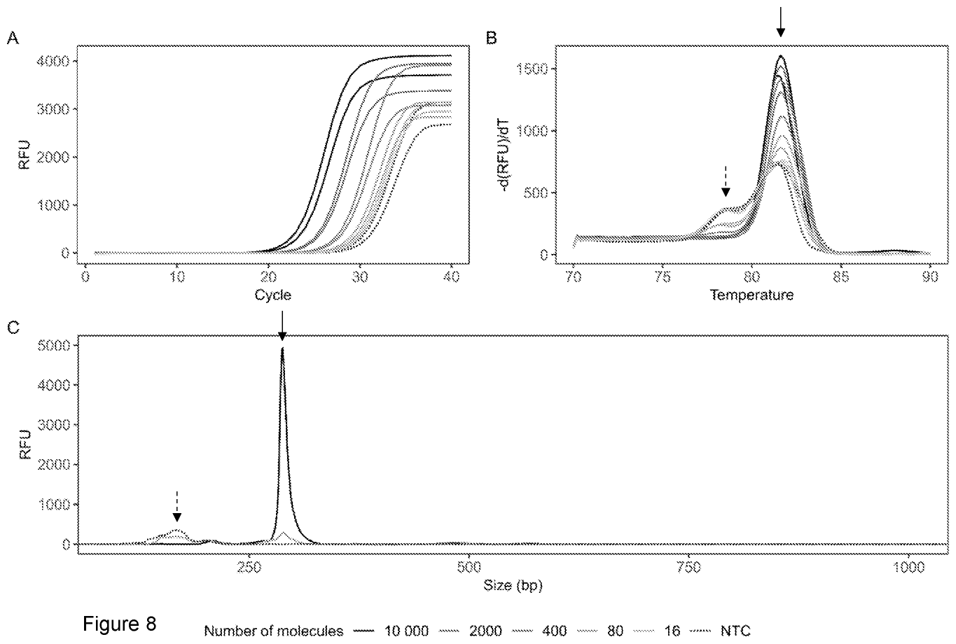

Next, the same target primers were tested but with hairpin protected UMI added (FIG. 3B). The formation of barcoded PCR-products of each primer pair combination was analyzed by qPCR followed by melting curve analysis using a standard curve, ranging from 10,000 to 16 molecules with 5-fold dilution steps. Quantitative PCR analysis showed that the dynamic range of all assays spanned the entire range, and the melting curve analysis indicated that specific PCR products were formed (FIGS. 8A and 8B). The presence of correct library size was also validated by Fragment Analyzer analysis (FIG. 8C). At lower DNA concentrations, the amount of non-specific PCR products increased, but specific PCR products were still generated. However, these DNA concentrations were far below relevant biological concentration for any practical applications using immune repertoire sequencing.

### Validation of a Simple, Robust and Fast Sequencing Protocol Targeting the TRD Gene

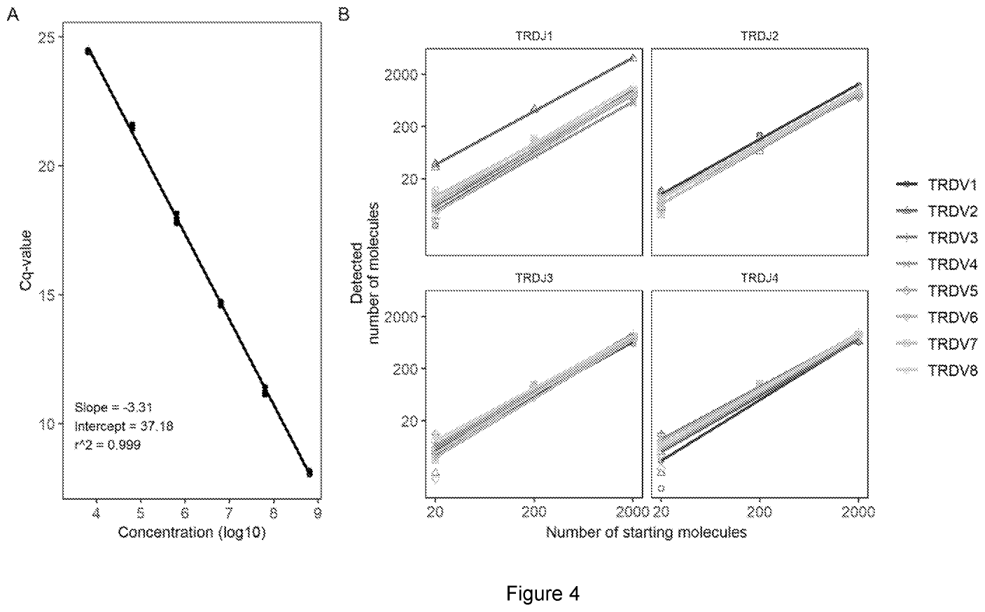

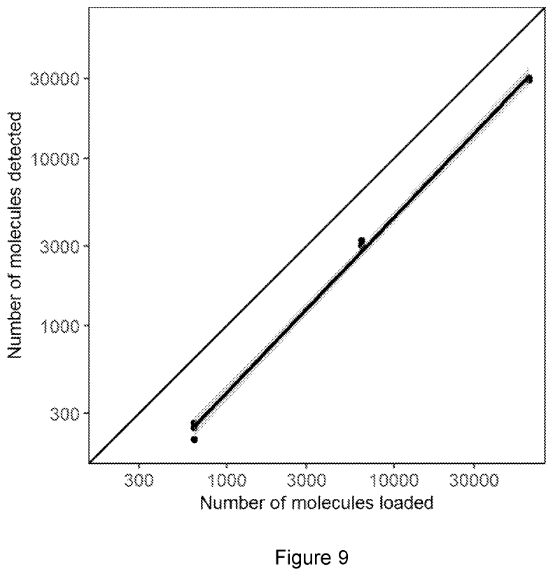

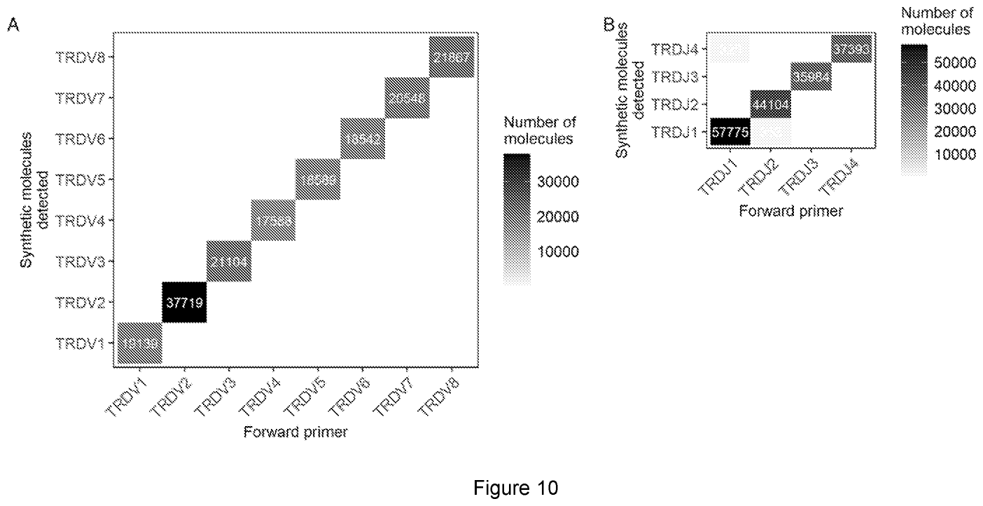

To determine amplification efficiency, sensitivity, and reproducibility of the final 32-plex assay targeting TRD gene rearrangements, standard curves of synthetic gBlock molecules were generated, ranging from 2×10⁷ to 20 molecules per target sequence. To test the overall PCR efficiency and dynamic range of the 32-plex assay, we first performed barcoding PCR and then quantified the barcoded PCR product with qPCR using the ILLUMINA® adapter primers. The overall PCR efficiency for the 32-plex assay was 101% (FIG. 4A). Next, the libraries generated from 2000, 200, and 20 synthetic reference DNA standard molecules were sequenced to evaluate the performance of each primer pair combination (FIG. 4B). The sequencing efficiency ranged between 98% and 117% for all assays (Table 5). The number of detected molecules for all assays together versus the number of starting molecules is shown in FIG. 9. As different TRDV and TRDJ genes share sequence homology we tested for off-target amplification. Synthetic gBlock DNA standards were matched by its unique template specific sequence to the primer that amplified respective molecule (FIG. 10). All TRDV primers showed high specificity where only 7 out of 175,103 (0.004%) barcoded families were amplified by the wrong TRDV target primer. For the TRDJ primers, the corresponding number was 705 out of 175,961 (0.4%) barcoded families, which was expected due to higher sequence similarity between J genes compared to V genes.

TABLE 5
Efficiency of immune repertoire sequencing using
synthetic gBlock molecules.

| | Target | Efficiency | |
|---|---|---|---|
| Target TRDV | TRDJ | (%) | R² |
| TRDV1 | TRDJ1 | 112.1 | 0.984 |
| TRDV1 | TRDJ2 | 105.7 | 0.994 |
| TRDV1 | TRDJ3 | 104.3 | 0.999 |
| TRDV1 | TRDJ4 | 117.1 | 0.947 |
| TRDV2 | TRDJ1 | 102.6 | 0.999 |
| TRDV2 | TRDJ2 | 98.4 | 0.988 |
| TRDV2 | TRDJ3 | 115.4 | 0.974 |
| TRDV2 | TRDJ4 | 107.7 | 0.960 |
| TRDV3 | TRDJ1 | 111.2 | 0.989 |
| TRDV3 | TRDJ2 | 107.0 | 0.991 |
| TRDV3 | TRDJ3 | 107.5 | 0.981 |
| TRDV3 | TRDJ4 | 101.1 | 0.992 |
| TRDV4/TRAV14 | TRDJ1 | 105.7 | 0.981 |
| TRDV4/TRAV14 | TRDJ2 | 104.0 | 0.999 |
| TRDV4/TRAV14 | TRDJ3 | 105.5 | 0.989 |
| TRDV4/TRAV14 | TRDJ4 | 108.3 | 0.988 |
| TRDV5/TRAV29 | TRDJ1 | 101.8 | 0.998 |
| TRDV5/TRAV29 | TRDJ2 | 105.2 | 0.997 |
| TRDV5/TRAV29 | TRDJ3 | 102.6 | 0.987 |
| TRDV5/TRAV29 | TRDJ4 | 109.4 | 0.976 |
| TRDV6/TRAV23 | TRDJ1 | 113.6 | 0.990 |
| TRDV6/TRAV23 | TRDJ2 | 100.0 | 0.997 |
| TRDV6/TRAV23 | TRDJ3 | 112.6 | 0.965 |
| TRDV6/TRAV23 | TRDJ4 | 101.7 | 0.996 |
| TRDV7/TRAV36 | TRDJ1 | 98.6 | 0.996 |
| TRDV7/TRAV36 | TRDJ2 | 110.7 | 0.991 |
| TRDV7/TRAV36 | TRDJ3 | 112.7 | 0.990 |
| TRDV7/TRAV36 | TRDJ4 | 108.3 | 0.991 |
| TRDV8/TRAV38-2 | TRDJ1 | 108.6 | 0.987 |
| TRDV8/TRAV38-2 | TRDJ2 | 104.3 | 0.996 |
| TRDV8/TRAV38-2 | TRDJ3 | 100.9 | 0.996 |
| TRDV8/TRAV38-2 | TRDJ4 | 106.5 | 0.997 |

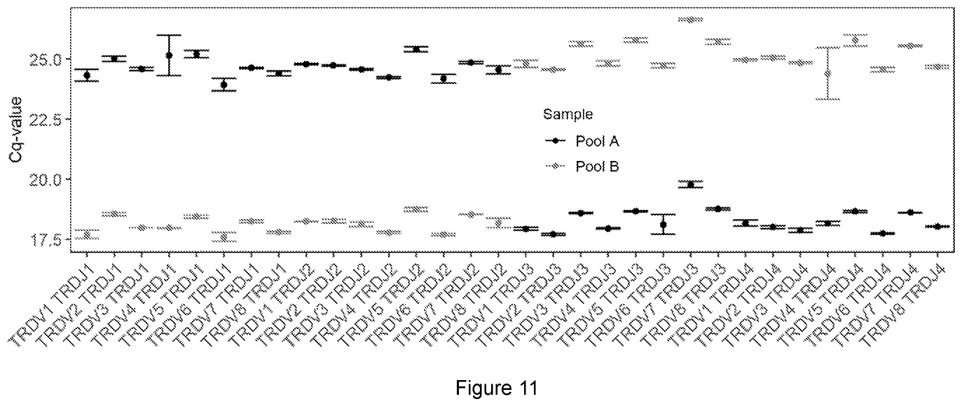

To further determine the sensitivity to detect rare TRD clones, pools of synthetic gBlock standards were generated where we sequenced 10 standard molecules per assay for 16 assays (160 molecules in total) in a background of 1000 standard molecules per assay for the remaining 16 individual assays (16,000 molecules in total). Hence, the sensitivity of each primer pair combination was tested at a ratio of 1:1616 (˜0.06%). The pools of synthetic gBlock molecules were quantified by qPCR (FIG. 11) and then by sequencing (FIG. 4C). Sequencing data showed that 1000, as well as 10 molecules, were reproducibly detected for all assays. Some variations between the assays may be explained by dilution artifacts as observed by qPCR (FIG. 11).

### Immune Repertoire Sequencing of TRD Using Enriched γδ T Cells

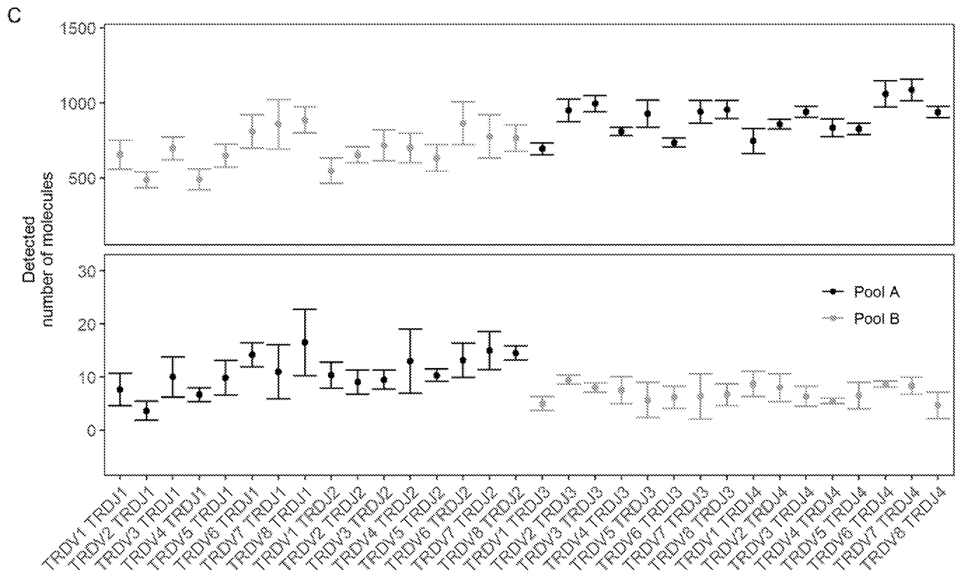

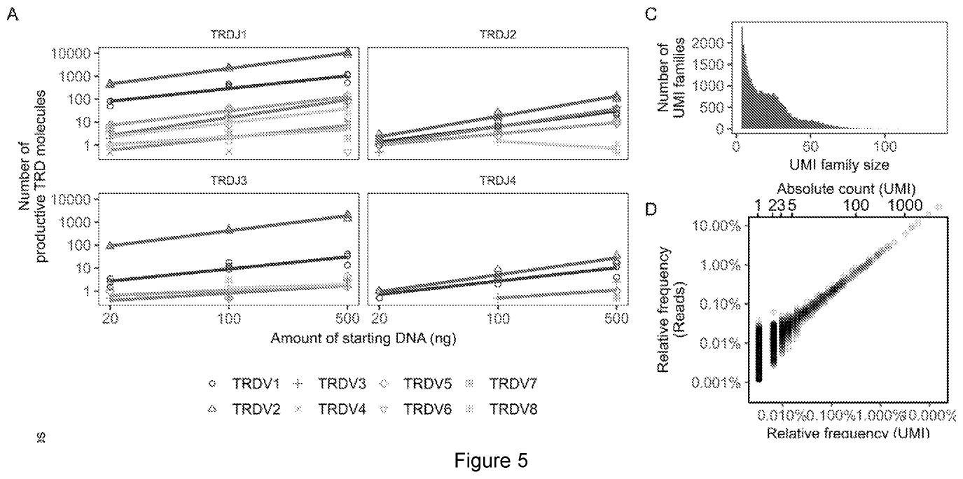

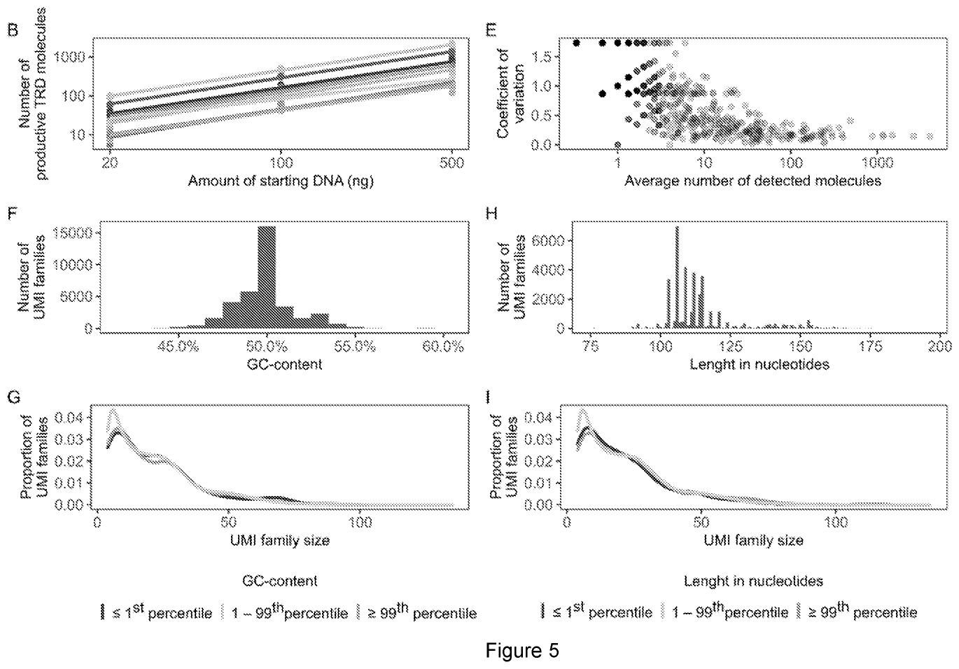

To validate our 32-plex assay on human samples, the genomic DNA extracted from γδ T cells enriched from buffy coats was analyzed using immunomagnetic cell separation. Sequencing libraries were constructed from 500, 100, and 25 ng γδ T-cell DNA and then sequenced. The total number of productive TRD molecules for each combination of rearranged TRD genes showed a linear correlation with the amount of analyzed DNA (FIG. 5A). FIG. 5B displays the ten most commonly produced clonotypes. One advantage of using UMIs is that it can correct for amplification biases between molecules. FIG. 5C shows the non-uniform amplification of different UMI families. Several specific UMIs were only observed once, while some specific UMIs were detected more than 100 times. This quantitative bias was corrected since all reads with the same UMI originated from the same molecule and were collapsed into one molecule. FIG. 5D illustrates the errors when not using UMI. For example, individual clonotypes with a single UMI sometimes displayed more than ten-fold variability in frequency when analyzed with raw sequencing reads. FIG. 5E shows the reproducibility when detecting distinct number of molecules using UMIs. The coefficient of variation increased when detecting rare clonotypes, which agrees with sampling ambiguity when detecting few molecules. Furthermore, no correlations were detected between reads per UMI and amplicon length but a small decrease of reads per UMI for high GC-content (FIGS. 5F-5I), indicating that the quantitative sequencing approach was largely independent of sequence context.

### Immune Repertoire Sequencing of TRD in Healthy Donors

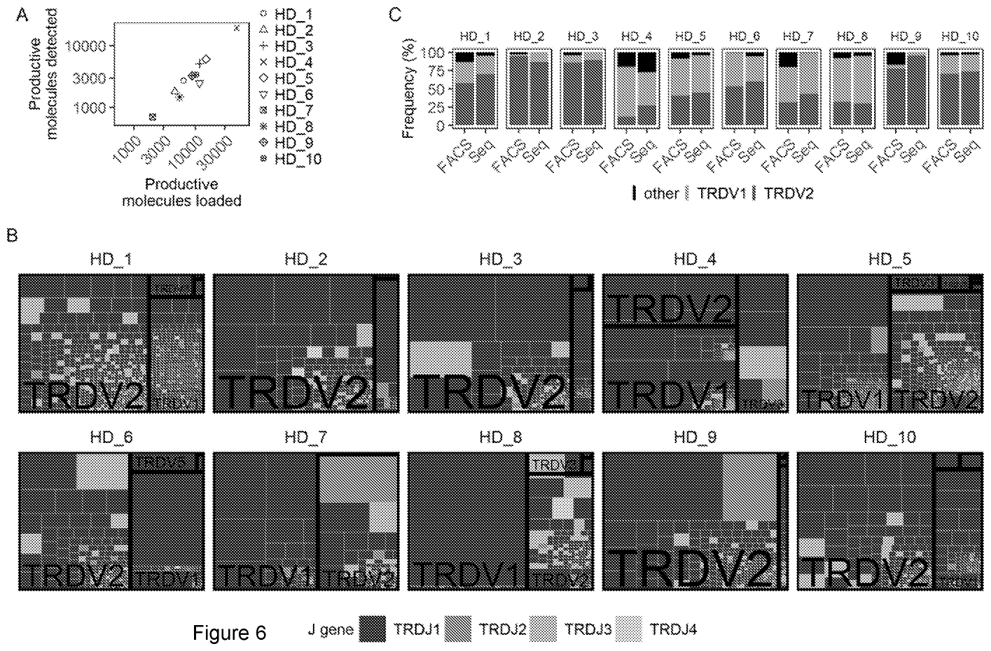

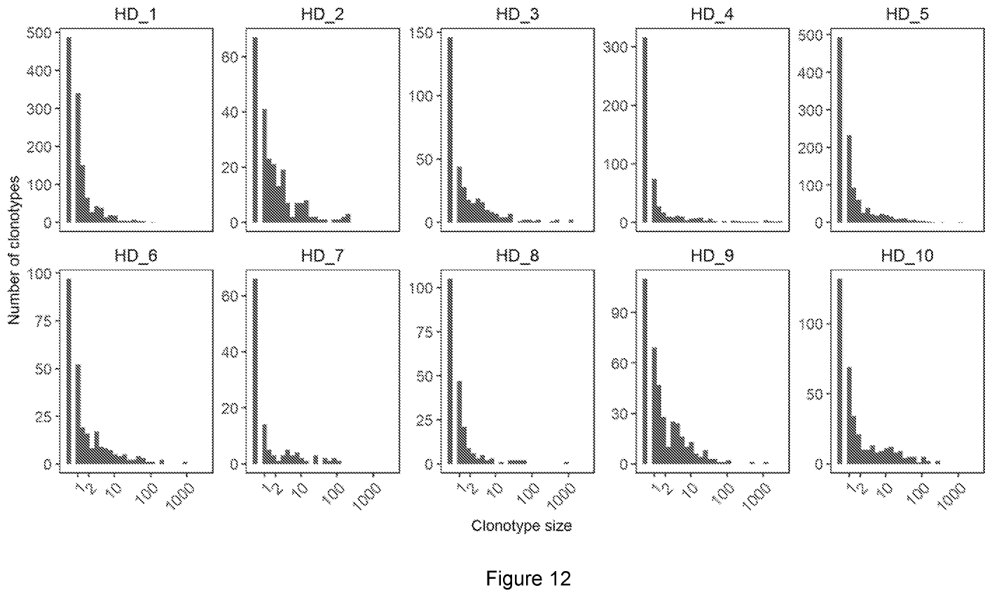

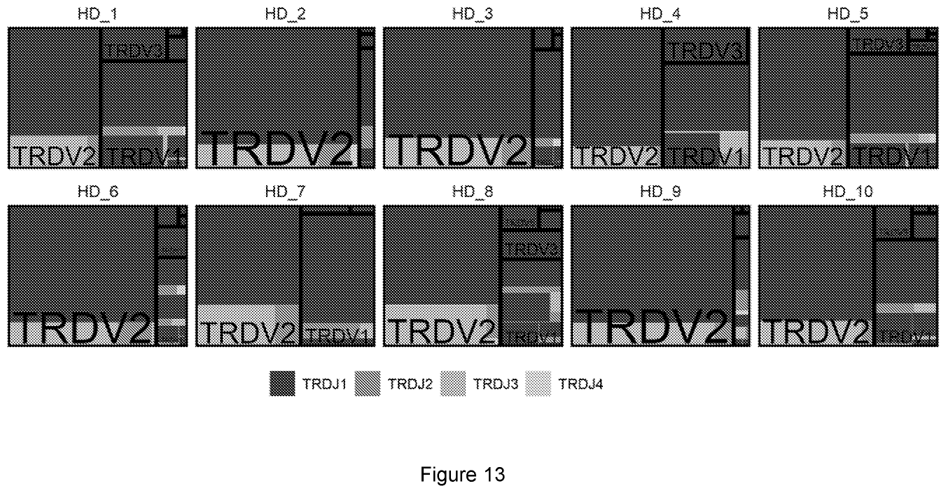

To characterize individual TRD immune repertoires, γδ T cells, enriched by fluorescence-activated cell sorting (FACS), were analyzed from PBMC of ten healthy individuals. The number of productive CDR3 sequences detected by sequencing correlated with the estimated amount of productive CDR3 γδ T-cell molecules loaded (FIG. 6A). The TRD repertoire diversity was different between the ten individuals (FIG. 6B). Donors 1 and 10 displayed no single clonotype with a frequency above 10%, while donors 3, 4, 6, 7, 8, and 9 were highly oligoclonal, where the top five clonotypes represented more than 50% of all productive TRD molecules detected. The distributions of clonotype sizes are shown in FIG. 12. The most common TRD rearrangement used among the different clonotypes was between TRDV2 and TRDJ1 (FIG. 13). To validate the genomic approach at the cellular level the frequencies of TRDV1 and TRDV2 were determined using FACS (FIG. 6C). The frequencies of TRDV1 and TRDV2 detected by FACS correlated significantly with immune repertoire sequencing.

### Immune Repertoire Sequencing Using Non-Enriched Cells

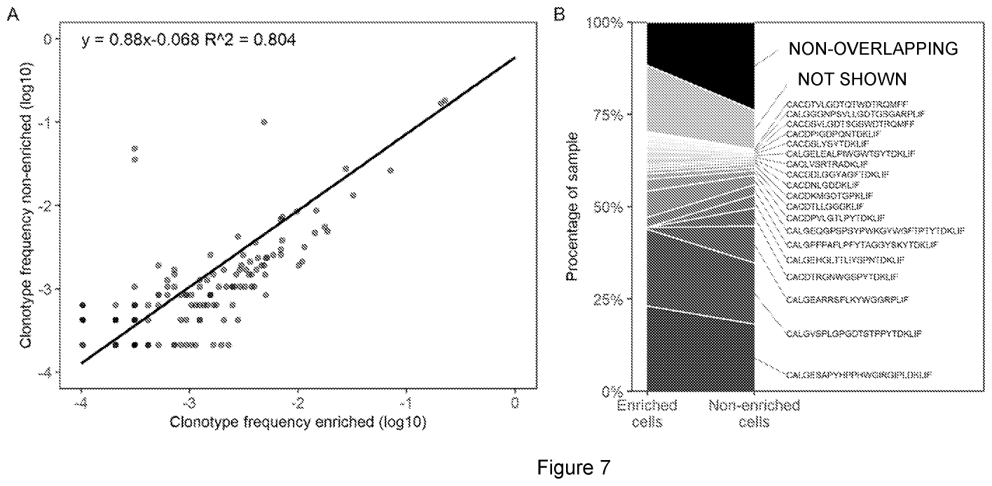

One major advantage of targeted PCR is that cell enrichment is potentially not needed. To compare sequencing from non-enriched PBMC and enriched γδ T cells, PBMC from a buffy coat were isolated and split into two equal aliquots: one sample was enriched for γδ T cells using negative selection with magnetic beads, while the other sample was analyzed directly without further cell enrichment. 100 ng DNA of the γδ T-cell enriched sample and 1 μg of the non-enriched sample were sequenced. In total, 9,925 and 4,836 productive TRD molecules were identified in the γδ T-cell enriched and the non-enriched samples, respectively (FIGS. 7A and 7B). The clonotype frequencies between non-enriched and enriched cells correlated linearly (R²=0.803). However, three outlier clonotypes were identified. These clonotypes were abundantly expressed in non-enriched cells, while their relative frequencies in enriched cells were low. A drawback of analyzing non-enriched γδ T cells is that partially rearranged loci and unproductively rearranged alleles of TRD in αβ T cells may be amplified. Indeed, the number of out-of-frame sequences was higher in non-enriched cells (44.5%) compared to enriched γδ T cells (15.6%).

### Discussion

A novel method for immune repertoire sequencing was developed to increase the quantification accuracy by multiple order of magnitudes for low-frequency clones. Improved quantification accuracy was achieved by counting the number of UMIs, which related to the original number of DNA molecules and, therefore, the number of cells. Consequently, PCR introduced amplification biases were avoided. The method minimized sequencing errors by the construction of consensus sequences. Compared to ultrasensitive detection of allele variants, such as mutation analysis, immune repertoire sequencing is experimentally more challenging since the sequences of individual target DNA molecules are different from each other, including variable GC-content and length. Allele variant analysis regularly only includes the detection of two different sequences, often with a single nucleotide variant. The 32-plex assay showed a small GC-content bias, but the error was corrected using UMIs as long as the original molecule that was amplified.

True clonal variation is difficult to separate from sequencing errors. By comparing the frequency of two clonotypes that differ by one or a few nucleotides, non-UMI based bioinformatical methods assume that the low-frequency clonotype is a sequencing mistake. This strategy is only applicable when another similar clone exists and is highly expressed, but still, this strategy may eliminate true low-frequency clones. The risk to misinterpret similar clonotypes is especially high for B cells that often display clonotypes with single nucleotide variations. The use of UMIs addressed all these issues, enabling accurate immune repertoire sequencing. PCR-based approaches that introduce UMIs correct for all DNA polymerase induced errors, except errors that occur in the first barcoding PCR step. These errors may be reduced by the use of high-fidelity polymerase. However, sequence errors that are not polymerase-induced, such as chemically modified bases, cannot be corrected by the use of UMIs. Another advantage with PCR-based approaches is the sensitivity when amplifying few target molecules. We demonstrate that it is possible to generate libraries from small amounts of DNA, at least as low as 15 ng for γδ T-cell derived DNA or less than 1000 synthetic molecules. The ability to detect small amounts of DNA is essential when analyzing tissues where the proportion of target γδ T-cell derived DNA is low. With a PCR-based approach, there is also no need for target cell enrichment. A strong correlation was shown between the frequencies of clonotypes detected in enriched and non-enriched samples. Interestingly, for the sample tested, three clonotypes were detected as outliers with more than a ten-fold higher frequency in the non-enriched sample. The reason for this is unknown, but one possibility may be that antibodies used in the depletion cocktail for negative selection also reacted to and depleted a subset of γγ T cells. Hence, immune repertoire DNA sequencing approaches without cell enrichments are not only simpler but may also be more accurately reflecting true clonotype frequencies.

The approach to add UMI by targeted PCR is both simpler and more sensitive than ligation-based methods. Sequence libraries can be generated within four hours, with limited hands-on time. Another advantage of PCR-based approaches is the possibility to choose a subset of target primers for specific applications. For example, in minimal residual disease in lymphoid malignancies where the immunoreceptor clonotype is known, and the detection of other immunoreceptor recombinations is of limited value, it is possible to monitor relevant clones longitudinally in a cost-effective manner.

### Example 2

In this Example ultrasensitive immune repertoire sequencing is used to explore the repertoire of the recombined immunoglobulin heavy (IGH) locus. IGH is a region on human chromosome 14 that contains a gene for the heavy chains of human antibodies (or immunoglobulins). The IGH locus includes V (variable), D (diversity), J (joining), and C (constant) segments.

### Materials and Methods

### Assay Design

Using a set of primers specific for all the IGH variable (V) regions and a different set of primers specific for all IGH joining (J) regions (Table 6) an immune repertoire can be amplified and analyzed using ultrasensitive immune repertoire sequencing. In this Example, the primer containing the hairpin with included barcode is the primer complementary to the J region. The primers are not perfectly complementary to the variable or joining region as each primer can amplify a plurality of variable or joining regions with share homology, respectively.

### Barcoding PCR and Library Construction

DNA was barcoded in a 10 μl reaction, containing 0.2 U Phusion HF polymerase, 1× Phusion High-Fidelity Buffer (both Thermo Fisher Scientific), 0.2 mM dNTP (Sigma-Aldrich), 0.5 M L-carnitine inner salt (Sigma-Aldrich), 40 nM of each barcode primer (PAGE purified, IDT) (Table 6) and 100 ng of DNA from B-cells. The following temperature program was used on a T100 Thermal cycler (BioRad): 98° C. for 30 sec, 3 cycles of amplification (98° C. for 10 sec, 62° C. for 6 min, 72° C. for 30 sec, all ramping rate of 4° C./sec), and 65° C. for 15 min and 95° C. for 15 min. Twenty microliters of 45 ng/μl Streptomyces griseus protease (Sigma-Aldrich) dissolved in TE-buffer (pH 8.0) was added to each well at the start of the 15 min incubation step at 65° C. to degrade the polymerase, reducing the formation of non-specific PCR products. ILLUMINA© adapters were added in a second PCR step (Table 3). Ten microliters of barcoded PCR product was amplified in a 40 μl reaction containing, 1×Q5 Hot Start High-Fidelity Master Mix (New England BioLabs) and 400 nM of each ILLUMINA© Adapter index primer (desalted, Sigma-Aldrich, Table 3) using the following thermal program on a T100 Thermal cycler; 98° C. for 3 min, to 40 cycles of amplification (98° C. for 10 sec, 80° C. for 1 sec, 72° C. for 30 sec, 76° C. for 30 sec, all ramping rate of 0.2° C./sec). Final reaction concentrations are shown for each PCR.

TABLE 6
primers for immune repertoire sequencing of the IGH locus

| Name | Sequence 5′-3′ | SEQ ID NO: |
|---|---|---|
| J-h- | GGACACTCTTTCCCTACACGACGCTCTTCCGATCT-UMI- | 83 |
| consensus_rev | ATGGGAAAGAGTGTCCCTTACCTGAGGAGACGGTGACC | |
| VH1-FR3_fw | GTGACTGGAGTTCAGACGTGTGCTCTTCCGATCTTGGAGCTGAGCAGC | 84 |
| | CTGAGATCTGA | |
| VH2-FR3_fw | GTGACTGGAGTTCAGACGTGTGCTCTTCCGATCTCAATGACCAACATG | 85 |
| | GACCCTGTGGA | |
| VH3-FR3_fw | GTGACTGGAGTTCAGACGTGTGCTCTTCCGATCTTCTGCAAATGAACA | 86 |
| | GCCTGAGAGCC | |
| VH4-FR3_fw | GTGACTGGAGTTCAGACGTGTGCTCTTCCGATCTGAGCTCTGTGACCG | 87 |
| | CCGCGGACACG | |
| VH5-FR3_fw | GTGACTGGAGTTCAGACGTGTGCTCTTCCGATCTCAGCACCGCCTACC | 88 |
| | TGCAGTGGAGC | |
| VH6-FR3_fw | GTGACTGGAGTTCAGACGTGTGCTCTTCCGATCTGTTCTCCCTGCAGC | 89 |
| | TGAACTCTGTG | |
| VH7-FR3_fw | GTGACTGGAGTTCAGACGTGTGCTCTTCCGATCTCAGCACGGCATATC | 90 |
| | TGCAGATGAG | |

Libraries were purified using the Agencourt AMPure XP system (Beckman Coulter), according to the manufacturer's instructions with a bead to sample ratio of 1:1. Library quality and quantification were assessed on a Fragment Analyzer using the HS NGS Fragment DNF-474 kit (both Agilent), according to the manufacturer's instructions.

Final quantification of library pool was performed by qPCR using NEBNext Library Quant Kit (New England Biolabs), according to manufacturer's instructions. Sequencing was performed on a Miniseq using midi output reagent KIT (300 cycles) with 20% added Phix control v3 (both ILLUMINA©) and 1 pM library. We performed 150 cycles of paired-end sequencing.

### Data Analysis

Sequencing data were processed using Molecular Identifier Guided Error Correction (MIGEC) pipeline using only paired-end reads and overlap-max-offset set to 100 (Shugay et al., 2014). In summary, UMIs were extracted from raw sequencing reads. Data were then grouped according to their UMI sequences and assembled to consensus reads with applied error correction. In the nomenclature used here, all corrected reads with identical sequences including the UMI form a UMI family that corresponds to one rearrangement and all UMI families with identical IGH sequence add up to a clonotype. CDR3 sequence was extracted from the consensus read and variable, diversity and joining segments were determined by the software. Results were filtered to only include productive IGH rearrangements.

### Results

### Immune Repertoire Sequencing of IGH Using Enriched B Cells

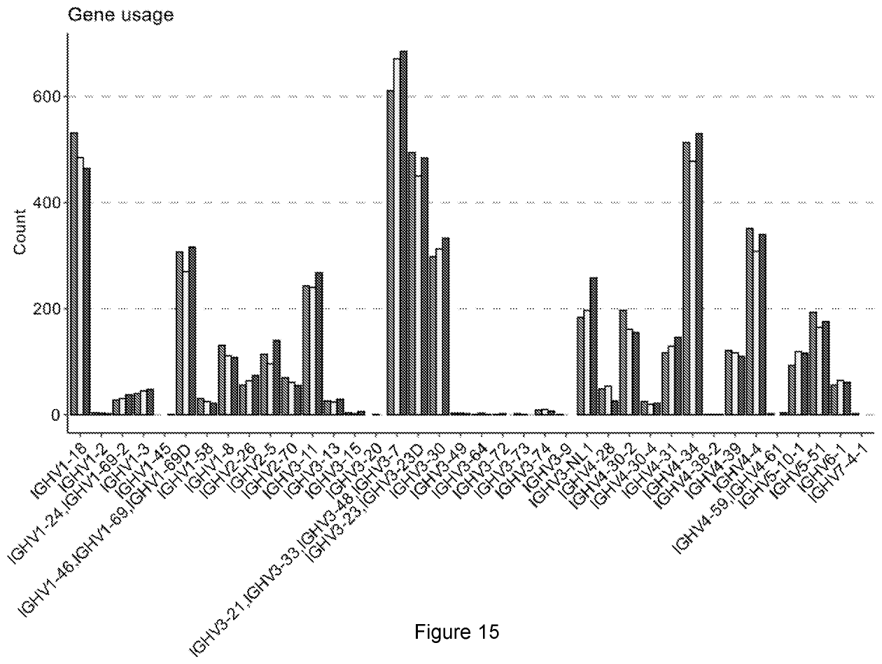

To validate the IGH assay on human samples, the genomic DNA extracted from B cells enriched using immunomagnetic cell separation from buffy coats was analyzed in tripplicates. Sequencing libraries were generated, sequenced, and analyzed bioinformatically as described in the material and methods. The total number of productive IGH molecules for each IGHV gene is shown in FIG. 15, indicating successful amplification of the IGH locus and low variation between the replicates.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

### REFERENCES

Gentles, A. J., Newman, A. M., Liu, C. L., Bratman, S. V., Feng, W., Kim, D., Nair, V. S., Xu, Y., Khuong, A., Hoang, C. D., et al. (2015). The prognostic landscape of genes and infiltrating immune cells across human cancers. Nat. Med. 21, 938-945.
Giudicelli, V., Chaume, D., and Lefranc, M.-P. P. (2005). IMGT/GENE-DB: A comprehensive database for human and mouse immunoglobulin and T cell receptor genes. Nucleic Acids Res. 33, 256-261.
Kent, W. J., Sugnet, C. W., Furey, T. S., Roskin, K. M., Pringle, T. H., Zahler, A. M., and Haussler, D. (2002). The human genome browser at UCSC. Genome Res. 12, 996-1006.
Nazarov, V. I., Pogorelyy, M. V., Komech, E. A., Zvyagin, I. V., Bolotin, D. A., Shugay, M., Chudakov, D. M., Lebedev, Y. B., and Mamedov, I. Z. (2015). tcR: an R package for T cell receptor repertoire advanced data analysis. BMC Bioinformatics 16, 175.
Shugay, M., Bagaev, D. V., Turchaninova, M. A., Bolotin, D. A., Britanova, 0. V., Putintseva, E. V., Pogorelyy, M. V., Nazarov, V. I., Zvyagin, I. V., Kirgizova, V. I., et al. (2015). VDJtools: Unifying Post-analysis of T Cell Receptor Repertoires. PLOS Comput. Biol. 11, el004503.
Singh, A. K., Novakova, L., Axelsson, M., Malmeström, C., Zetterberg, H., Lycke, J., and Cardell, S. L. (2017). High interferon-γ uniquely in Vδ1 T cells correlates with markers of inflammation and axonal damage in early multiple sclerosis. Front. Immunol. 8.
Smith, T., Heger, A., Sudbery, I., (2017). UMI-tools: modeling sequencing errors in Unique Molecular Identifiers to improve quantification accuracy. Genome Research 27, 491-499.
Stahlberg, A., Krzyzanowski, P. M., Jackson, J. B., Egyud, M., Stein, L., Godfrey, T. E., Stehlberg, A., Krzyzanowski, P. M., Jackson, J. B., Egyud, M., et al. (2016). Simple, multiplexed, PCR-based barcoding of DNA enables sensitive mutation detection in liquid biopsies using sequencing. Nucleic Acids Res. 44, e105.
St\dot{a}hlberg, A., Krzyzanowski, P. M., Egyud, M., Filges, S., Stein, L., and Godfrey, T. E. (2017). Simple multiplexed PCR-based barcoding of DNA for ultrasensitive mutation detection by next-generation sequencing. Nat. Protoc. 12, 664-682.
Ye, J., Coulouris, G., Zaretskaya, I., Cutcutache, I., Rozen, S., and Madden, T. L. (2012). Primer-BLAST: A tool to design target-specific primers for polymerase chain reaction. BMC Bioinformatics 13, 134.
Zarobkiewicz, M. K., Kowalska, W., Rolinski, J., and Bojarska-Junak, A. A. (2019). γδ T lymphocytes in the pathogenesis of multiple sclerosis and experimental autoimmune encephalomyelitis. J. Neuroimmunol. 330, 67-73.

1.-20. (canceled)

21. A method of determining lymphocyte clonality, the method comprising:
contacting a sample comprising nucleic acid molecules of lymphocytes with M forward primers and N reverse primers, wherein
M is an integer equal to or larger than one and Nis an integer equal to or larger than one;
the M forward primers comprise, from a 5′ end to a 3′ end, an adapter sequence and a target-specific sequence;
the N reverse primers comprise, from a 5′ end to a 3′ end, an adapter sequence and a target-specific sequence;
the M forward primers are M hairpin barcode forward primers and/or the N reverse primers are N hairpin barcode reverse primers;
each hairpin barcode forward primer comprises, from the 5′ end to the 3′ end, a 5′ stem sequence, the adapter sequence, a unique molecular identifier (UMI), a 3′ stem sequence and the target-specific sequence complementary to a respective portion of a T-cell receptor (TCR) or B-cell receptor (BCR) clonotype;
each hairpin barcode reverse primer comprises, from the 5′ end to the 3′ end, a 5′ stem sequence, the adapter sequence, a UMI, a 3′ stem sequence and the target-specific sequence complementary to a respective portion of a TCR or BCR clonotype; and
at least a portion of the 5′ stem sequence of the hairpin barcode forward primer and/or the hairpin barcode reverse primer is complementary to at least a portion of the 3′ stem sequence of the hairpin barcode forward primer and/or the hairpin barcode reverse primer, the 5′ stem sequence and the 3′ stem sequence are configured to hybridize to each other at or under a closed annealing temperature and not hybridize to each other at or above an open annealing temperature;
amplifying the nucleic acid molecules by performing polymerase chain reaction (PCR) pre-amplification of the nucleic acid molecules to form a plurality of barcoded PCR products, wherein the PCR pre-amplification has an annealing temperature equal to or less than the closed annealing temperature of the hairpin barcode forward primers and/or the hairpin barcode reverse primers;
contacting the plurality of barcoded PCR products with an adapter-specific forward primer and an adapter-specific reverse primer;
amplifying the barcoded PCR products by performing PCR amplification on the barcoded PCR products to form a library of amplified barcoded PCR products, wherein at least a portion of cycles of the PCR amplification has an annealing temperature equal to or greater than the open annealing temperature of the hairpin barcode forward primers and/or the hairpin barcode reverse primer;
sequencing at least a respective portion of the amplified barcoded PCR products to form respective sequence reads comprising the UMI(s) and TCR or BCR sequence(s);
demultiplexing the sequence reads based on nucleic acid sequences of the UMIs;
mapping the demultiplexed sequence reads to respective TCR or BCR clonotypes based on nucleic acid sequences of the TCR or BCR sequences; and
determining lymphocyte clonality for the sample based on the demultiplexed and mapped sequence reads.

22. The method according to claim 21, wherein amplifying the nucleic acid molecules comprises amplifying the nucleic acid molecules by performing 1-25 cycles of PCR pre-amplification of the nucleic acid molecules to form the plurality of barcoded PCR products.

23. The method according to claim 22, wherein amplifying the nucleic acid molecules comprises amplifying the nucleic acid molecules by performing 2-20 cycles of PCR pre-amplification of the nucleic acid molecules to form the plurality of barcoded PCR products.

24. The method according to claim 23, wherein amplifying the nucleic acid molecules comprises amplifying the nucleic acid molecules by performing 2-15 cycles of PCR pre-amplification of the nucleic acid molecules to form the plurality of barcoded PCR products.

25. The method according to claim 21, wherein amplifying (S4) the barcoded PCR products comprises amplifying the barcoded PCR products by performing at least 2 cycles of PCR amplification on the barcoded PCR products to form a library of amplified barcoded PCR products.

26. The method according to claim 25, wherein amplifying (S4) the barcoded PCR products comprises amplifying the barcoded PCR products by performing at least 3 cycles of PCR amplification on the barcoded PCR products to form a library of amplified barcoded PCR products.

27. The method according to claim 26, wherein amplifying (S4) the barcoded PCR products comprises amplifying the barcoded PCR products by performing at least 5 cycles of PCR amplification on the barcoded PCR products to form a library of amplified barcoded PCR products.

28. The method according to claim 21, wherein demultiplexing the sequence reads comprises dividing the sequence reads into groups having a same nucleotide sequence of the UMIs, optionally with at most a predefined number of mismatches allowed for nucleotide sequences of UMIs in a same group.

29. The method according to claim 21, wherein mapping the demultiplexed sequence reads comprises dividing demultiplexed sequence reads into groups having a same nucleotide sequence of the TCR or BCR sequences, with at most a predefined number of mismatches allowed for nucleotide sequences of TCR or BCR sequences in a same group.

30. The method according to claim 21, wherein determining lymphocyte clonality comprises quantifying the TCR or BCR clonotypes present in the sample based on the demultiplexed and mapped sequence reads.

31. The method according to claim 30, wherein quantifying the TCR or BCR clonotypes comprises quantifying TCR or BCR clonotypes based on determining the number of different UMIs, with at most a first predefined number of mismatches, having the same nucleotide sequence of the TCR or BCR sequence, with at most a second predefined number of mismatches.

32. The method according to claim 21, further comprising degrading a polymerase used for amplifying the nucleic acid molecules in the PCR pre-amplification prior to amplifying the barcoded PCR products in the PCR amplification.

33. The method according to claim 21, wherein the 3′ stem sequence comprises 5-15 nucleotides.

34. The method according to claim 33, wherein the 3′ stem sequence comprises 8-15 nucleotides.

35. The method according to claim 34, wherein the 3′ stem sequence comprises 12-15 nucleotides.

36. The method according to claim 21, wherein the hairpin barcode forward primer and/or the hairpin barcode reverse primer further comprises at least one destabilizing nucleotide between the UMI and the 3′ stem sequence.

37. The method according to claim 36, wherein the hairpin barcode forward primer and/or the hairpin barcode reverse primer further at least two destabilizing nucleotides between the UMI and the 3′ stem sequence.

38. The method according to claim 21, wherein the UMI is a random n₁n₂n₃ . . . nₖ sequence, wherein nᵢ, i=1 . . . k, is one of A, T, C and G, and k is from 6 up to 18.

39. The method according to claim 38, wherein the UMI is a random n₁n₂n₃ . . . nₖ sequence, wherein nᵢ, i=1 . . . k, is one of A, T, C and G, and k is from 10 up to 15.

40. The method according to claim 39, wherein the UMI is a random n₁n₂n₃ . . . nₖ sequence, wherein nᵢ, i=1 . . . k, is one of A, T, C and G, and k is 12.

41. The method according to claim 21, wherein
the adapter-specific forward primer comprises, from a 5′ end to a 3′ end, one of a P5 sequence and a P7 sequence and a sequence equal to or complementary to the adapter sequence of the M forward primers; and
the adapter-specific reverse primer comprises, from a 5′ end to a 3′ end, the other of the P5 sequence and the P7 sequence and a sequence equal to or complementary to the adapter sequence of the N reverse primer.

42. The method according to claim 41, wherein
the P5 sequence preferably comprises AATGATACGGCGACCACCGA (SEQ ID NO: 15) or AATGATACGGCGACCACCGAGATCTACAC (SEQ ID NO: 51); and
the P7 sequence comprises CAAGCAGAAGACGGCATACGAGAT (SEQ ID NO: 16).

43. The method according to claim 42, wherein the adapter sequence of the M forward primers is different from the adapter sequence of the N reverse primer.

44. The method according to claim 21, wherein the closed annealing temperature is equal to or less than 65° C.

45. The method according to claim 21, wherein the open annealing temperature is at least 70° C.

46. The method according to claim 21, wherein if M is one then Nis equal to or larger than two and if Nis one then M is equal to or larger than two.

47. The method according to claim 21, wherein
the M forward primers are multiple different hairpin barcode forward primers comprising at least one hairpin barcode forward primer per variable (V) region of a TCR or BCR; and
each target-specific sequence of the hairpin barcode forward primers is complementary to a respective variable (V) region of the TCR or BCR.

48. The method according to claim 47, wherein contacting the sample comprises contacting the sample with the multiple different hairpin barcode forward primers and multiple different reverse primers, wherein
the multiple different reverse primers comprise at least one reverse primer per joining (J) region of the TCR or BCR; and
each reverse primer comprises, from the 5′ end to the 3′ end, the adapter sequence and a target-specific sequence complementary to a respective joining (J) region of the TCR or BCR.

49. The method according to claim 21, wherein
the N reverse primers is a hairpin barcode reverse primer comprising a target-specific sequence complementary to a joining (J) region of an immunoglobulin heavy chain (IGH) of the BCR; and
the M forward primers comprise at least one forward primer per variable (V) region of the IGH of the BCR.

50. A method of disease characterization, the method comprising:
determining lymphocyte clonality according to claim 21 of a sample comprising nucleic acid molecules of lymphocytes obtained from a subject; and
characterizing a disease of the subject based on the determined lymphocyte clonality.

51. The method according to claim 50, wherein the disease is selected from the group consisting of a hematologic disease, an infectious disease, a cancer disease and an autoimmune disease.


SEQUENCE LISTING (90 sequences)

SEQ ID NO: 1 - DNA, 20 bp, Homo sapiens
gcgaaatccgtcgccttaac

SEQ ID NO: 2 - DNA, 23 bp, Homo sapiens
acttgcaccatcagagagagatg

SEQ ID NO: 3 - DNA, 23 bp, Homo sapiens
tccagtaaggactgaagacagtg

SEQ ID NO: 4 - DNA, 21 bp, Homo sapiens
ccagaaggcaagaaaatccgc

SEQ ID NO: 5 - DNA, 23 bp, Homo sapiens
cttaaacaaaagtgccaagcacc

SEQ ID NO: 6 - DNA, 23 bp, Homo sapiens
gcagttctcatcgcatatcatgg

SEQ ID NO: 7 - DNA, 19 bp, Homo sapiens
agaccggagactcggccat

SEQ ID NO: 8 - DNA, 18 bp, Homo sapiens
ggggatgccgcgatgtat

SEQ ID NO: 9 - DNA, 20 bp, Homo sapiens
cacagtcacacgggttcctt

SEQ ID NO: 10 - DNA, 24 bp, Homo sapiens
cgatgagttgtgttccctttccaa

SEQ ID NO: 11 - DNA, 21 bp, Homo sapiens
agtttgatgccagttccgaaa

SEQ ID NO: 12 - DNA, 23 bp, Homo sapiens
gttgtacctccagataggttcct

SEQ ID NO: 13 - DNA, 63 bp, Artificial Sequence
  Hairpin barcode forward primer
  misc_feature (36)..(47): n is a, c, g, or t
ggacactctttccctacacgacgctcttccgatctnnnnnnnnnnnnatgggaaagagtgtcc

SEQ ID NO: 14 - DNA, 34 bp, Artificial Sequence
  Reverse primer
gtgactggagttcagacgtgtgctcttccgatct

SEQ ID NO: 15 - DNA, 20 bp, Artificial Sequence
  ILLUMINA P5 sequence
aatgatacggcgaccaccga

SEQ ID NO: 16 - DNA, 24 bp, Artificial Sequence
  ILLUMINA P7 sequence
caagcagaagacggcatacgagat

SEQ ID NO: 17 - DNA, 58 bp, Artificial Sequence
  Universal forward adapter primer
aatgatacggcgaccaccgagatctacactctttccctacacgacgctcttccgatct

SEQ ID NO: 18 - DNA, 64 bp, Artificial Sequence
  Reverse index 1 adapter primer
caagcagaagacggcatacgagatcgtgatgtgactggagttcagacgtgtgctcttccgatct

SEQ ID NO: 19 - DNA, 64 bp, Artificial Sequence
  Reverse index 2 adapter primer
caagcagaagacggcatacgagatacatcggtgactggagttcagacgtgtgctcttccgatct

SEQ ID NO: 20 - DNA, 64 bp, Artificial Sequence
  Reverse index 3 adapter primer
caagcagaagacggcatacgagatgcctaagtgactggagttcagacgtgtgctcttccgatct

SEQ ID NO: 21 - DNA, 64 bp, Artificial Sequence
  Reverse index 4 adapter primer
caagcagaagacggcatacgagattggtcagtgactggagttcagacgtgtgctcttccgatct

SEQ ID NO: 22 - DNA, 64 bp, Artificial Sequence
  Reverse index 5 adapter primer
caagcagaagacggcatacgagatcactgtgtgactggagttcagacgtgtgctcttccgatct

SEQ ID NO: 23 - DNA, 64 bp, Artificial Sequence
  Reverse index 6 adapter primer
caagcagaagacggcatacgagatattggcgtgactggagttcagacgtgtgctcttccgatct

SEQ ID NO: 24 - DNA, 64 bp, Artificial Sequence
  Reverse index 7 adapter primer
caagcagaagacggcatacgagatgatctggtgactggagttcagacgtgtgctcttccgatct

SEQ ID NO: 25 - DNA, 64 bp, Artificial Sequence
  Reverse index 8 adapter primer
caagcagaagacggcatacgagattcaagtgtgactggagttcagacgtgtgctcttccgatct

SEQ ID NO: 26 - DNA, 64 bp, Artificial Sequence
  Reverse index 9 adapter primer
caagcagaagacggcatacgagatctgatcgtgactggagttcagacgtgtgctcttccgatct

SEQ ID NO: 27 - DNA, 64 bp, Artificial Sequence
  Reverse index 10 adapter primer
caagcagaagacggcatacgagataagctagtgactggagttcagacgtgtgctcttccgatct

SEQ ID NO: 28 - DNA, 252 bp, Artificial Sequence
  Synthetic gBlock molecule
tcggtggttctctaactactatcaccagcaaaatgcaacagaaggtcgctactcattgaatttccagaaggcaagaaaatccgccaaccttgtcatctccgcttcacaactgggggactcagcaatgtacttctgtgcaatgagagagggagctagctaggttgtgtactagctagctagacaccgataaactcatctttggaaaaggaacccgtgtgactgtggaaccaaccaacgaacatgtaactctca

SEQ ID NO: 29 - DNA, 255 bp, Artificial Sequence
  Synthetic gBlock molecule
tcggtggttctctaactactatcaccagcaaaatgcaacagaaggtcgctactcattgaatttccagaaggcaagaaaatccgccaaccttgtcatctccgcttcacaactgggggactcagcaatgtacttctgtgcaatgagagagggagctagctagaaatcggtctagctagctagctttgacagcacaactcttctttggaaagggaacacaactcatcgtggaaccagccaacgaacatgtaactctca

SEQ ID NO: 30 - DNA, 260 bp, Artificial Sequence
  Synthetic gBlock molecule
tcggtggttctctaactactatcaccagcaaaatgcaacagaaggtcgctactcattgaatttccagaaggcaagaaaatccgccaaccttgtcatctccgcttcacaactgggggactcagcaatgtacttctgtgcaatgagagagggagctagctagtatgcctgaaagctagctagctcctgggacacccgacagatgtttttcggaactggcatcaaactcttcgtggagccccccaacgaacatgtaactctca

SEQ ID NO: 31 - DNA, 249 bp, Artificial Sequence
  Synthetic gBlock molecule
tcggtggttctctaactactatcaccagcaaaatgcaacagaaggtcgctactcattgaatttccagaaggcaagaaaatccgccaaccttgtcatctccgcttcacaactgggggactcagcaatgtacttctgtgcaatgagagagggagctagctagatgggtcattagctagctagccagacccctgatctttggcaaaggaacctatctggaggtacaacaacccaacgaacatgtaactctca

SEQ ID NO: 32 - DNA, 270 bp, Artificial Sequence
  Synthetic gBlock molecule
tcggtggttctctaactactatcttattgatagccatacgtccagatgtgagtgaaaagaaagaaggaagattcacaatctccttcaataaaagtgccaagcagttctcatcgcatatcatggattcccagcctggagactcagccacctacttctgtgcagcaagcaagctagctagcccctttagtagctagctagacaccgataaactcatctttggaaaaggaacccgtgtgactgtggaaccaaccaacgaacatgtaactctca

SEQ ID NO: 33 - DNA, 273 bp, Artificial Sequence
  Synthetic gBlock molecule
tcggtggttctctaactactatcttattgatagccatacgtccagatgtgagtgaaaagaaagaaggaagattcacaatctccttcaataaaagtgccaagcagttctcatcgcatatcatggattcccagcctggagactcagccacctacttctgtgcagcaagcaagctagctaggaaatcgtagagctagctagctttgacagcacaactcttctttggaaagggaacacaactcatcgtggaaccagccaacgaacatgtaactctca

SEQ ID NO: 34 - DNA, 278 bp, Artificial Sequence
  Synthetic gBlock molecule
tcggtggttctctaactactatcttattgatagccatacgtccagatgtgagtgaaaagaaagaaggaagattcacaatctccttcaataaaagtgccaagcagttctcatcgcatatcatggattcccagcctggagactcagccacctacttctgtgcagcaagcaagctagctagtcattttgcgagctagctagctcctgggacacccgacagatgtttttcggaactggcatcaaactcttcgtggagccccccaacgaacatgtaactctca

SEQ ID NO: 35 - DNA, 267 bp, Artificial Sequence
  Synthetic gBlock molecule
tcggtggttctctaactactatcttattgatagccatacgtccagatgtgagtgaaaagaaagaaggaagattcacaatctccttcaataaaagtgccaagcagttctcatcgcatatcatggattcccagcctggagactcagccacctacttctgtgcagcaagcaagctagctagggtgtgcaaaagctagctagccagacccctgatctttggcaaaggaacctatctggaggtacaacaacccaacgaacatgtaactctca

SEQ ID NO: 36 - DNA, 242 bp, Artificial Sequence
  Synthetic gBlock molecule
tcggtggttctctaactactatcataaaaatgaagatggaagattcactgttttcttaaacaaaagtgccaagcacctctctctgcacattgtgccctcccagcctggagactctgcagtgtacttctgtgcagcaagcgagctagctagaattctatttagctagctagacaccgataaactcatctttggaaaaggaacccgtgtgactgtggaaccaaccaacgaacatgtaactctca

SEQ ID NO: 37 - DNA, 245 bp, Artificial Sequence
  Synthetic gBlock molecule
tcggtggttctctaactactatcataaaaatgaagatggaagattcactgttttcttaaacaaaagtgccaagcacctctctctgcacattgtgccctcccagcctggagactctgcagtgtacttctgtgcagcaagcgagctagctagtattttgcttagctagctagctttgacagcacaactcttctttggaaagggaacacaactcatcgtggaaccagccaacgaacatgtaactctca

SEQ ID NO: 38 - DNA, 250 bp, Artificial Sequence
  Synthetic gBlock molecule
tcggtggttctctaactactatcataaaaatgaagatggaagattcactgttttcttaaacaaaagtgccaagcacctctctctgcacattgtgccctcccagcctggagactctgcagtgtacttctgtgcagcaagcgagctagctagccggccggggagctagctagctcctgggacacccgacagatgtttttcggaactggcatcaaactcttcgtggagccccccaacgaacatgtaactctca

SEQ ID NO: 39 - DNA, 239 bp, Artificial Sequence
  Synthetic gBlock molecule
tcggtggttctctaactactatcataaaaatgaagatggaagattcactgttttcttaaacaaaagtgccaagcacctctctctgcacattgtgccctcccagcctggagactctgcagtgtacttctgtgcagcaagcgagctagctagggttaccctcagctagctagccagacccctgatctttggcaaaggaacctatctggaggtacaacaacccaacgaacatgtaactctca

SEQ ID NO: 40 - DNA, 232 bp, Artificial Sequence
  Synthetic gBlock molecule
tcggtggttctctaactactatcgtcaggaagactaagtagcatattagataagaaagaacttttcagcatcctgaacatcacagccacccagaccggagactcggccatctacctctgtgctgtggaggagctagctagtttaagttaaagctagctagacaccgataaactcatctttggaaaaggaacccgtgtgactgtggaaccaaccaacgaacatgtaactctca

SEQ ID NO: 41 - DNA, 236 bp, Artificial Sequence
  Synthetic gBlock molecule
tcggtggttctctaactactatcgtcaggaagactaagtagcatattagataagaaagaacttttcagcatcctgaacatcacagccacccagaccggagactcggccatctacctctgtgctgtggaggagctagctagtgtactcaggcagctagctagctttgacagcacaactcttctttggaaagggaacacaactcatcgtggaaccagccaacgaacatgtaactctca

SEQ ID NO: 42 - DNA, 240 bp, Artificial Sequence
  Synthetic gBlock molecule
tcggtggttctctaactactatcgtcaggaagactaagtagcatattagataagaaagaacttttcagcatcctgaacatcacagccacccagaccggagactcggccatctacctctgtgctgtggaggagctagctagtcatgctgggagctagctagctcctgggacacccgacagatgtttttcggaactggcatcaaactcttcgtggagccccccaacgaacatgtaactctca

SEQ ID NO: 43 - DNA, 229 bp, Artificial Sequence
  Synthetic gBlock molecule
tcggtggttctctaactactatcgtcaggaagactaagtagcatattagataagaaagaacttttcagcatcctgaacatcacagccacccagaccggagactcggccatctacctctgtgctgtggaggagctagctagctcggtgcaaagctagctagccagacccctgatctttggcaaaggaacctatctggaggtacaacaacccaacgaacatgtaactctca

SEQ ID NO: 44 - DNA, 233 bp, Artificial Sequence
  Synthetic gBlock molecule
tcggtggttctctaactactatcagaatcgtttctctgtgaacttccagaaagcagccaaatccttcagtctcaagatctcagactcacagctgggggatgccgcgatgtatttctgtgcttataggagcgagctagctaggaaagggttcagctagctagacaccgataaactcatctttggaaaaggaacccgtgtgactgtggaaccaaccaacgaacatgtaactctca

SEQ ID NO: 45 - DNA, 236 bp, Artificial Sequence
  Synthetic gBlock molecule
tcggtggttctctaactactatcagaatcgtttctctgtgaacttccagaaagcagccaaatccttcagtctcaagatctcagactcacagctgggggatgccgcgatgtatttctgtgcttataggagcgagctagctagtttttacccaagctagctagctttgacagcacaactcttctttggaaagggaacacaactcatcgtggaaccagccaacgaacatgtaactctca

SEQ ID NO: 46 - DNA, 241 bp, Artificial Sequence
  Synthetic gBlock molecule
tcggtggttctctaactactatcagaatcgtttctctgtgaacttccagaaagcagccaaatccttcagtctcaagatctcagactcacagctgggggatgccgcgatgtatttctgtgcttataggagcgagctagctaggcccttttggagctagctagctcctgggacacccgacagatgtttttcggaactggcatcaaactcttcgtggagccccccaacgaacatgtaactctca

SEQ ID NO: 47 - DNA, 230 bp, Artificial Sequence
  Synthetic gBlock molecule
tcggtggttctctaactactatcagaatcgtttctctgtgaacttccagaaagcagccaaatccttcagtctcaagatctcagactcacagctgggggatgccgcgatgtatttctgtgcttataggagcgagctagctagattttttcccagctagctagccagacccctgatctttggcaaaggaacctatctggaggtacaacaacccaacgaacatgtaactctca

SEQ ID NO: 48 - DNA, 14 bp, Artificial Sequence
  5′ stem sequence
ggacactctttccc

SEQ ID NO: 49 - DNA, 14 bp, Artificial Sequence
  3′ stem sequence
gggaaagagtgtcc

SEQ ID NO: 50 - DNA, 33 bp, Artificial Sequence
  Adapter sequence
acactctttccctacacgacgctcttccgatct

SEQ ID NO: 51 - DNA, 29 bp, Artificial Sequence
  ILLUMINA P5 sequence
aatgatacggcgaccaccgagatctacac

SEQ ID NO: 52 - protein, 20 aa, Homo sapiens
CACDTVLGDTQTWDTRQMFF

SEQ ID NO: 53 - protein, 24 aa, Homo sapiens
CALGGGNPSVLLGDTGSGARPLIF

SEQ ID NO: 54 - protein, 21 aa, Homo sapiens
CACDSVLGDTSGSWDTRQMFF

SEQ ID NO: 55 - protein, 17 aa, Homo sapiens
CACDPIGDPQNTDKLIF

SEQ ID NO: 56 - protein, 15 aa, Homo sapiens
CACDSLYSYTDKLIF

SEQ ID NO: 57 - protein, 23 aa, Homo sapiens
CALGELEALPIWGWTSYTDKLIF

SEQ ID NO: 58 - protein, 15 aa, Homo sapiens
CACLVSRTRADKLIF

SEQ ID NO: 59 - protein, 18 aa, Homo sapiens
CACDDLGGYAGFTDKLIF

SEQ ID NO: 60 - protein, 13 aa, Homo sapiens
CACDNLGDDKLIF

SEQ ID NO: 61 - protein, 15 aa, Homo sapiens
CACDKMGDTGPKLIF

SEQ ID NO: 62 - protein, 14 aa, Homo sapiens
CACDTLLGGDKLIF

SEQ ID NO: 63 - protein, 18 aa, Homo sapiens
CACDPVLGTLPYTDKLIF

SEQ ID NO: 64 - protein, 30 aa, Homo sapiens
CALGEQGPSPSYPWKGYWGFTPTYTDKLIF

SEQ ID NO: 65 - protein, 27 aa, Homo sapiens
CALGPFPAFLPFYTAGGYSKYTDKLIF

SEQ ID NO: 66 - protein, 22 aa, Homo sapiens
CALGEHGLTTLIYSPNTDKLIF

SEQ ID NO: 67 - protein, 19 aa, Homo sapiens
CACDTRGNWGSPYTDKLIF

SEQ ID NO: 68 - protein, 21 aa, Homo sapiens
CALGEARRSFLKYWGGRPLIF

SEQ ID NO: 69 - protein, 24 aa, Homo sapiens
CALGVSPLGPGDTSTPPYTDKLIF

SEQ ID NO: 70 - protein, 26 aa, Homo sapiens
CALGESAPYHPPHWGIRGIPLDKLIF

SEQ ID NO: 71 - DNA, 222 bp, Homo sapiens
tcggtggttctctaactactatcctgtcaacttcaagaaagcagcgaaatccgtcgccttaaccatttcagccttacagctagaagattcagcaaagtacttttgtgctcttggggaactagctagctagaagatagaatagctagctagacaccgataaactcatctttggaaaaggaacccgtgtgactgtggaaccaaccaacgaacatgtaactctca

SEQ ID NO: 72 - DNA, 224 bp, Homo sapiens
tcggtggttctctaactactatcctgtcaacttcaagaaagcagcgaaatccgtcgccttaaccatttcagccttacagctagaagattcagcaaagtacttttgtgctcttggggaactagctagctaggtcaaactgagctagctagctttgacagcacaactcttctttggaaagggaacacaactcatcgtggaaccagccaacgaacatgtaactctca

SEQ ID NO: 73 - DNA, 228 bp, Homo sapiens
tcggtggttctctaactactatcctgtcaacttcaagaaagcagcgaaatccgtcgccttaaccatttcagccttacagctagaagattcagcaaagtacttttgtgctcttggggaactagctagctagttatcgatcagctagctagctcctgggacacccgacagatgtttttcggaactggcatcaaactcttcgtggagccccccaacgaacatgtaactctc

SEQ ID NO: 74 - DNA, 218 bp, Homo sapiens
tcggtggttctctaactactatcctgtcaacttcaagaaagcagcgaaatccgtcgccttaaccatttcagccttacagctagaagattcagcaaagtacttttgtgctcttggggaactagctagctagatcgtactgagctagctagccagacccctgatctttggcaaaggaacctatctggaggtacaacaacccaacgaacatgtaactctca

SEQ ID NO: 75 - DNA, 223 bp, Homo sapiens
tcggtggttctctaactactatcttccaaggtgacattgatattgcaaagaacctggctgtacttaagatacttgcaccatcagagagagatgaagggtcttactactgtgcctgtgacaccagctagctagggatcgactagctagctagacaccgataaactcatctttggaaaaggaacccgtgtgactgtggaaccaaccaacgaacatgtaactctca

SEQ ID NO: 76 - DNA, 226 bp, Homo sapiens
tcggtggttctctaactactatcttccaaggtgacattgatattgcaaagaacctggctgtacttaagatacttgcaccatcagagagagatgaagggtcttactactgtgcctgtgacaccagctagctagcatgattcaagctagctagctttgacagcacaactcttctttggaaagggaacacaactcatcgtggaaccagccaacgaacatgtaactctca

SEQ ID NO: 77 - DNA, 231 bp, Homo sapiens
tcggtggttctctaactactatcttccaaggtgacattgatattgcaaagaacctggctgtacttaagatacttgcaccatcagagagagatgaagggtcttactactgtgcctgtgacaccagctagctagaaatcgtaaagctagctagctcctgggacacccgacagatgtttttcggaactggcatcaaactcttcgtggagccccccaacgaacatgtaactctca

SEQ ID NO: 78 - DNA, 220 bp, Homo sapiens
tcggtggttctctaactactatcttccaaggtgacattgatattgcaaagaacctggctgtacttaagatacttgcaccatcagagagagatgaagggtcttactactgtgcctgtgacaccagctagctagttacgtactagctagctagccagacccctgatctttggcaaaggaacctatctggaggtacaacaacccaacgaacatgtaactctca

SEQ ID NO: 79 - DNA, 268 bp, Homo sapiens
tcggtggttctctaactactatctgtaaacaaatgaaactacttacagaaaaacaaaaagctaaagcccacagtaagaatcatcttttcttcatatcagggcagaggatatacaacaaaacagggttcctaagatctcagagactatctgacggttctaatgaaagaagctagctagacctgatgtagctagctagacaccgataaactcatctttggaaaaggaacccgtgtgactgtggaaccaaccaacgaacatgtaactctca

SEQ ID NO: 80 - DNA, 271 bp, Homo sapiens
tcggtggttctctaactactatctgtaaacaaatgaaactacttacagaaaaacaaaaagctaaagcccacagtaagaatcatcttttcttcatatcagggcagaggatatacaacaaaacagggttcctaagatctcagagactatctgacggttctaatgaaagaagctagctagtagcatgcaagctagctagctttgacagcacaactcttctttggaaagggaacacaactcatcgtggaaccagccaacgaacatgtaactctca

SEQ ID NO: 81 - DNA, 276 bp, Homo sapiens
tcggtggttctctaactactatctgtaaacaaatgaaactacttacagaaaaacaaaaagctaaagcccacagtaagaatcatcttttcttcatatcagggcagaggatatacaacaaaacagggttcctaagatctcagagactatctgacggttctaatgaaagaagctagctagcagtcactaagctagctagctcctgggacacccgacagatgtttttcggaactggcatcaaactcttcgtggagccccccaacgaacatgtaactctca

SEQ ID NO: 82 - DNA, 265 bp, Homo sapiens
tcggtggttctctaactactatctgtaaacaaatgaaactacttacagaaaaacaaaaagctaaagcccacagtaagaatcatcttttcttcatatcagggcagaggatatacaacaaaacagggttcctaagatctcagagactatctgacggttctaatgaaagaagctagctagatcgatgctagctagctagccagacccctgatctttggcaaaggaacctatctggaggtacaacaacccaacgaacatgtaactctca

SEQ ID NO: 83 - DNA, 85 bp, Artificial Sequence
  Reverse hairpin barcode primer
  misc_feature (36)..(47): n is a, c, g, or t
ggacactctttccctacacgacgctcttccgatctnnnnnnnnnnnnatgggaaagagtgtcccttacctgaggagacggtgacc

SEQ ID NO: 84 - DNA, 59 bp, Artificial Sequence
  Forward primer
gtgactggagttcagacgtgtgctcttccgatcttggagctgagcagcctgagatctga

SEQ ID NO: 85 - DNA, 59 bp, Artificial Sequence
  Forward primer
gtgactggagttcagacgtgtgctcttccgatctcaatgaccaacatggaccctgtgga

SEQ ID NO: 86 - DNA, 59 bp, Artificial Sequence
  Forward primer
gtgactggagttcagacgtgtgctcttccgatcttctgcaaatgaacagcctgagagcc

SEQ ID NO: 87 - DNA, 59 bp, Artificial Sequence
  Forward primer
gtgactggagttcagacgtgtgctcttccgatctgagctctgtgaccgccgcggacacg

SEQ ID NO: 88 - DNA, 59 bp, Artificial Sequence
  Forward primer
gtgactggagttcagacgtgtgctcttccgatctcagcaccgcctacctgcagtggagc

SEQ ID NO: 89 - DNA, 59 bp, Artificial Sequence
  Forward primer
gtgactggagttcagacgtgtgctcttccgatctgttctccctgcagctgaactctgtg

SEQ ID NO: 90 - DNA, 58 bp, Artificial Sequence
  Forward primer
gtgactggagttcagacgtgtgctcttccgatctcagcacggcatatctgcagatcag